(19) United States
(12) Patent Application Publication
(10) Pub. No.: US 2024/0344094 A1
(43) Pub. Date: Oct. 17, 2024

(54) METHODS AND COMPOSITIONS USEFUL FOR THE PRODUCTION OF OLEFINIC ESTER

(71) Applicant: THE REGENTS OF THE UNIVERSITY OF CALIFORNIA, Oakland, CA (US)

(72) Inventors: Taek Soon Lee, Berkeley, CA (US); David Carruthers, Oakland, CA (US); Jinho Kim, Emeryville, CA (US); Daniel Mendez-Perez, Shrewsbury, MA (US); Eric Sundstrom, San Mateo, CA (US)

(73) Assignee: THE REGENTS OF THE UNIVERSITY OF CALIFORNIA, Oakland, CA (US)

(21) Appl. No.: 18/644,618

(22) Filed: Apr. 24, 2024

Related U.S. Application Data
(63) Continuation of application No. PCT/US2022/078805, filed on Oct. 27, 2022.
(60) Provisional application No. 63/341,670, filed on May 13, 2022.
(60) Provisional application No. 63/272,593, filed on Oct. 27, 2021.

(51) Int. Cl. C12P 7/62; C12N 1/20; C12N 9/10; C12N 9/16; C12R 1/19

(52) U.S. Cl. CPC C12P 7/62; C12N 1/205; C12N 9/1029; C12N 9/16; C12R 2001/19; C12Y 203/01084; C12Y 301/03001

(57) ABSTRACT

The present invention provides for a method for producing an olefinic ester, the method comprising: (a) providing a host cell capable of producing olefinic ester; and (b) culturing the host cell to produce olefinic ester. In some embodiments, the olefinic ester is isoprenyl acetate. In some embodiments, the olefinic ester is isoprenyl acetate, and the method further comprises: (c) optionally recovering the isoprenyl acetate; (d) optionally (i) deacetylating the isoprenyl acetate into isoprene, and/or (ii) hydrolyzing isoprenyl acetate into isoprenol and dehydrating the isoprenol into isoprene; and, (e) optionally converting the isoprene into 1,4-dimethylcyclooctane (DMCO).

### CROSS-REFERENCE TO RELATED APPLICATIONS

This application claims priority to, and is a 35 U.S.C. § 111(a) continuation of, PCT international application number PCT/US2022/078805 filed on Oct. 27, 2022, incorporated herein by reference in its entirety, which claims priority to, and the benefit of, U.S. provisional patent application Ser. No. 63/341,670 filed on May 13, 2022, incorporated herein by reference in its entirety, and which also claims priority to, and the benefit of, U.S. provisional patent application Ser. No. 63/272,593 filed on Oct. 27, 2021, incorporated herein by reference in its entirety. Priority is claimed to each of the foregoing applications.

The above-referenced PCT international application was published as PCT International Publication No. WO 2023/077017 A1 on May 4, 2023, which publication is incorporated herein by reference in its entirety.

### STATEMENT OF GOVERNMENTAL SUPPORT

The invention described and claimed herein was made utilizing funds supplied by the U.S. Department of Energy under Contract No. DE-AC02-05CH11231. The government has certain rights in this invention.

### INCORPORATION-BY-REFERENCE OF SEQUENCE LISTING

This application includes a sequence listing in an xml file entitled “UCLBL-2021-141-04-US-seq-listing.xml” created on Jan. 24, 2023 and having a 14 kb file size. The sequence listing is submitted electronically through Patent Center and is incorporated herein by reference in its entirety.

### FIELD OF THE INVENTION

The present invention is in the field of production of producing olefinic ester.

### BACKGROUND OF THE INVENTION

Current sources of isoprenyl acetate stem from esterification of isoprenol (3-methyl-3 butenol) with acetyl chloride, acetic anhydride, or a like reactant. Isoprenol is generated by a palladium catalyzed reaction of petrochemically derived isobutene and formaldehyde.

### SUMMARY OF THE INVENTION

The present invention provides for a method for producing an olefinic ester, the method comprising: (a) providing a host cell capable of producing olefinic ester; and (b) culturing the host cell to produce olefinic ester.

In some embodiments, the olefinic ester is isoprenyl acetate. In some embodiments, the olefinic ester is isoprenyl acetate, and the method further comprises: (c) optionally recovering the isoprenyl acetate; (d) optionally (i) deacetylating the isoprenyl acetate into isoprene, and/or (ii) hydrolyzing isoprenyl acetate into isoprenol and dehydrating the isoprenol into isoprene; and, (e) optionally converting the isoprene into 1,4-dimethyliyclooctane (DMCO).

In some embodiments, the host cell is capable of producing mevalonate (MVA) monophosphate (MVAP), and comprises a mevalonate diphosphate decarboxylase (PMD), or homologous enzyme thereof, and an alcohol acyltransferase (ATF1), or homologous enzyme thereof. In some embodiments, the host cell is natively capable of producing MVAP, or the host cell is natively capable of producing MVAP and is genetically modified to increase the producing of MVAP, or the host cell is natively not capable of producing MVAP and is genetically modified to produce MVAP. In some embodiments, the host cell is genetically modified by the introduction of acetyl-CoA acetyltransferase (AtoB), hydroxymethylglutaryl-CoA synthase (MvaS or HMGS), hydroxymethylglutaryl-CoA reductase (MvaA or HMGR), and/or mevalonate kinase (MK), homologous enzyme(s) thereof.

In some embodiments, the PMD, or homologous enzyme thereof, comprises an amino acid sequence having at least about 70%, 75%, 80%, 85%, 90%, 95%, or 99% amino acid sequence identity with SEQ ID NO:1 or SEQ ID NO:2. In some embodiments, the ATF1, or homologous enzyme thereof, comprises an amino acid sequence having at least about 70%, 75%, 80%, 85%, 90%, 95%, or 99% amino acid sequence identity with SEQ ID NO:3, SEQ ID NO:4, SEQ ID NO:5, or SEQ ID NO:6.

The present invention provides for genetically modified host cells, such as an engineered E. coli strain, that can produce esters of isoprenoid derived alcohols. In some embodiments, the genetically modified host cells, such as the engineered E. coli strain, are modified via episomal expression of a heterologous mevalonate pathway with the isopentenyl diphosphate (IPP) bypass and a promiscuous phosphatase to generate isoprenol. In some embodiments, further expression of an alcohol acyltransferase, ATF1, catalyzes the acetylation of isoprenol and acetyl-CoA to generate isoprenyl acetate, a high value chemical used as a fuel, fragrance, or food additive with significant market potential. High titer production has been demonstrated in laboratory scale batch and fed-batch 2 L reactors on glucose. This strain has demonstrated the highest yet reported titer of isoprenyl acetate with an approximately 100-fold improvement over current means. In some embodiments, the platform can be expanded to include different alcohols, such as the isomer prenol for prenyl acetate production, and to include other carboxylic acid-CoA thioesters including, but not limited to, lactyl-CoA, propionyl-CoA, and dimethylacrylyl-CoA. In some embodiments, the platform could also be modified for growth on other carbon substrates like pretreated lignocellulosic hydrolysate.

In some embodiments, the genetically modified host cell has the expression of pta, poxB, and/or ackA reduced, knocked out, or deleted or knocked out, or a nucleic acid encoding the pta, poxB, and/or ackA operatively linked to an inducible promoter. In some embodiments, the genetically modified host cell has its endogenous expression of pta, poxB, and/or ackA reduced, knocked out, or deleted or knocked out, or a nucleic acid encoding the pta, poxB, and/or ackA operatively linked to an inducible promoter or a promoter with a transcriptional strength lower than the native promoters thereof.

Typically, deletion of pta, poxB, and ackA result in near complete loss of acetate production, which would ideally drive an accumulation of acetyl-CoA. Increased expression of Acetyl-CoA synthetases (ACS) increases conversion of acetate into acetyl-CoA substrate. In some embodiments, the host cell comprises an acetyl-CoA synthetases (ACS), or homologous enzyme thereof. In some embodiments, the host cell is genetically modified by the introduction of an ACS, or homologous enzyme thereof.

In some embodiments, the ACS, or homologous enzyme thereof, comprises an amino acid sequence having at least about 70%, 75%, 80%, 85%, 90%, 95%, or 99% amino acid sequence identity with one of SEQ ID NOs:7-9. SEQ ID NO:9 is the amino acid sequence of Salmonella enterica ACS with an L641P point mutation that reduces acetylation and feedback inhibition, thereby continually converting acetate into acetyl-CoA precursor.

In some embodiments, the ACS, or homologous enzyme thereof, comprises one or more, or all, of the amino acid residues conserved or having identity between E. coli and Salmonella enterica ACS. In some embodiments, the ACS comprises the conserved residues of SEQ ID Nos:7 and 8 in positions 311, 335, 523, and 584 which are important for binding Coenzyme A. In some embodiments, the ACS comprises the conserved residues of SEQ ID Nos:7 and 8 in positions 500, 515, and 526 which are important for binding ATP. In some embodiments, the ACS comprises the conserved residues of SEQ ID Nos:7 and 8 in positions 537, 539, and 542 which are important for binding magnesium. In some embodiments, the ACS comprises the amino acid sequences GEP at positions 387-389 and DTWWQT (SEQ ID NO:10) at positions 411-416, which are important for binding the ATP nucleotide.

In some embodiments, the genetically modified host cell is a host cell or microorganism, or comprises one or more aspects thereof, disclosed by U.S. Pat. Nos. 7,985,567; 9,631,210; 10,273,506; and, 10,814,724; and, PCT International Patent Application Nos. PCT/US2008/068831 and PCT/US2012/055165 (herein incorporated by reference).

3-methyl-3-buten-1-ol (isoprenol) can be chemically converted into DMCO. This conversion has been described, wherein isoprene is used as a precursor for DMCO by a two step chemical process—dimerization and hydrogenation (Rosenkoetter et al. Green Chem., 2019), isoprene, however, has several issues to be used as a biosynthetic precursor for DMCO such as a risk of explosion. Isoprene can also be produced from the dehydration of isoprenol, and therefore isoprenol has been employed as an alternate precursor for DMCO as it is safer for large scale production and for transportation than isoprene.

Isoprenol has a solubility of about 90 g/L (20° (7) in water and at this solubility level, it could be toxic to the production host such as E. coli or S. cerevisiae. The esters of isoprenol such as isoprenyl acetate offer significant production advantages over isoprenol, including enhanced partitioning, reduced product toxicity, and higher volatility. Upon the confirmation that the conversion of isoprenyl acetate to DMCO can be achieved at a similar efficiency to that of isoprenol to DMCO, isoprenyl acetate will be a good alternate precursor of DMCO.

This present invention is based on a recombinant strain of E. coli with episomal expression of the IPP-bypass pathway taught by Kang and Lee (U.S. Pat. Nos. 10,273,506; and, 10,814,724). The IPP-bypass pathway capitalizes upon the promiscuity of a modified mevalonate diphosphate decarboxylase (PMD) in a heterologously expressed mevalonate pathway. The mutant PMD enables generation of isoprenyl phosphate (IP) directly from mevalonate diphosphate thereby bypassing the toxic intermediate, IPP. Kang and Lee demonstrated enhanced production of isoprenol using AphA, a promiscuous phosphatase.

Isoprenyl acetate is produced through acetylation of isoprenol with acetyl-CoA and is catalyzed by an acetyltransferase favoring primary alcohol substrates. Building upon the work of Kang and Lee, this invention included a yeast acyltransferase, ATF1, which enables high efficiency, in vivo esterification of isoprenol to isoprenyl acetate. Production of the high titer cultures required an investigation of other acyltransferases including ATF2 as well as considerable culture optimization with various organic overlays, media composition, and ambient culture conditions. The ATF described herein was found to demonstrate high titer production of isoprenyl acetate. Further acyltransferases suitable for the invention include the following enzymes: chloramphenicol acetyltransferase (Zada et al., Biotechnology for Biofuels, 2018), Fragaria anonassa AAT (Lee et al., Biotechnology for Biofuels, 2020), and other AATs known to act on downstream isoprenoids like geraniol and farnesol (Latimer et al., ACS Synthetic Biology, 2020). The platform could also be modularly paired with varied aceyltransferases exhibiting favorable kinetics towards other CoA thioester moiety substrates. These include lactyl-CoA (Lee et al. Biotechnology for biofuels 2019), propionyl-CoA, and dimethylacrylate-CoA (Eiben et al. ACS Synthetic Biology, 2020) to yield prenyl or isoprenyl lactate, prenyl or isoprenyl propionate, and ethyl 3,3-dimethylacrylate, respectively. Similar to isoprenyl acetate, these esters maintain favorable properties as fuel additives. The best bioproduction publication (Zada et al., Biotechnology for Biofuels, 2018) used the MVA pathway with the primary goal of generating longer chain prenyl diphosphates (C10 to C15) and which only produced isoprenyl acetate as a side product. The method and host cells of the present invention can be extrapolated to the production of other olefinic esters by varying intracellular accumulation of alternative carboxylic acid-CoA thioester.

It has been demonstrated that the present invention can be practiced in a high titer laboratory-scale production of isoprenyl acetate cultured in 2-L bioreactors with common and inexpensive laboratory medium. The invention can be further developed for practice in a 300-L, 1000-L, or larger, laboratory reactors for scale-up.

Current culture schemes also employ an organic overlay, such as an unsaturated fatty alcohol, such as oleyl alcohol, as a mechanism for sequestering the ester product from aqueous solution. Although the overlay facilitates chemical extraction, its application may not be feasible at scale. Likewise, further analysis must be conducted to assess the relative feasibility of downstream separation technologies for acceptable isolation of pure product. In some embodiments, the unsaturated fatty alcohol has the following chemical structure: α-CR═CR′-β-OH, wherein α and β are each independently a C₁ to C₂₀ alkyl, and R, and R′ are each independently H or a C₁ to C₂₀ alkyl, wherein 1, 2, 3, or 4 of α, β, R, and R′ comprises one or more hydroxyl groups. In some embodiments, α and β are each independently a C₅ to C₂₀ alkyl. In some embodiments, R and R′ are each independently H or a C₅ to C₂₀ alkyl. In some embodiments, the unsaturated fatty alcohol is an unsaturated fatty alcohol described in Egan et al. “Properties and uses of some unsaturated fatty alcohols and their derivatives,” J. Amer. Oil Chm. Soc., 61(2): 324-329, 1984 (herein incorporated by reference). Oleyel alcohol has the following chemical structure:

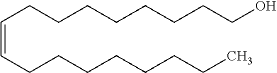 [SMILES: CCCCCCCC/C=C\CCCCCCCCO]

The isoprenyl acetate can be used as a food and fragrance additive, however as an olefinic ester it has a high relative octane number and thus favorable chemical properties as a gasoline additive. In addition, isoprenyl acetate is an alternative precursor of a potential jet fuel compound DMCO. A chemical route to convert isoprenol to DMCO has been established and isoprenyl acetate is an alternative to isoprenol in this reaction.

The present invention has the capacity to generate isoprenyl acetate at orders of magnitude higher than those offered by current commercial processes at substantially lower cost. Although isoprenol is relatively inexpensive, it is significantly more environmentally harmful than comparable bioproduction pathways as it is derived from petrochemical precursors. The biologically derived production strategy here is devoid of direct petrochemical contributions and instead is derived from glucose, Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Titer of isoprenyl acetate and cell density under batch fermentation conditions using M9 minimal media with yeast extract without oleyl alcohol overlay. Fermentations were run at 30° C. with 20 g/L of glucose and 5 g/L of yeast extract in the batch medium. ▪, isoprenyl acetate; ♦, triangle, cell density (OD₆₀₀).

FIG. 1B. Titer of isoprenyl acetate and cell density under batch fermentation conditions using M9 minimal media with yeast extract with oleyl alcohol overlay. Fermentations were run at 30° C. with 20 g/L of glucose and 5 g/L of yeast extract in the batch medium. ▪, isoprenyl acetate; ♦, triangle, cell density (OD₆₀₀).

FIG. 1C. Titer of isoprenyl acetate and cell density under fed-batch fermentation conditions using M9 minimal media and yeast extract without oleyl alcohol overlay. Fermentations were run at 30° C. with 20 g/L of glucose and 5 g/L of yeast extract in the fed-batch medium. 20 g/L of glucose and 1.25 g/L of yeast extract were fed at 24 h, 48 h, 72 h, and 96 h. ▪, isoprenyl acetate; ♦, cell density (OD₆₀₀); •, consumed glucose.

FIG. 1D. Titer of isoprenyl acetate and cell density by E. coli DH1 (JBEI-137161) under fed-batch fermentation conditions using M9 minimal media with an oleyl alcohol overlay. Fermentations were run at 30° C. with 20 g/L glucose in the fed-batch medium. 20 g/L glucose and 1.25 g/L NH₄Cl was fed at 24 h, 48 h, 72 h, and 96 h. A maximum titer of 15.0±0.9 g/L isoprenyl acetate was achieved.

FIG. 1E. Titer of isoprenyl acetate and cell density by E. coli DH1 (JBEI-137161+pBbS5k-Acs-se) under fed-batch fermentation conditions using M9 minimal media with an oleyl alcohol overlay. Fermentations were run at 30° C. with 20 g/L glucose in the fed-batch medium. 20 g/L glucose and 1.25 g/L NH₄Cl was fed at 24 h, 48 h, 72 h, and 96 h. A maximum titer of 21.1±0.2 g/L isoprenyl acetate was achieved.

FIG. 1F. Titer of isoprenyl acetate and cell density by E. coli DH1 (JBEI-3606+JBEI-137161) under fed-batch fermentation conditions using M9 minimal media with an oleyl alcohol overlay. Fermentations were run at 30° C. with 20 g/L glucose in the fed-batch medium. 20 g/L glucose and 1.25 g/L NH₄Cl was fed at 24 h, 48 h, 72 h, and 96 h. A maximum titer of 25.0±1.5 g/L isoprenyl acetate was achieved.

FIG. 1G. Titer of isoprenyl acetate and cell density by E. coli DH1 (JBEI-3606+JBEI-137161+pBbS5k-Acs-se) under fed-batch fermentation conditions using M9 minimal media with an oleyl alcohol overlay. Fermentations were run at 30° C. with 20 g/L glucose in the fed-batch medium. 20 g/L glucose and 1.25 g/L NH₄Cl was fed at 24 h, 48 h, 72 h, and 96 h. A maximum titer of 28.0 g/L isoprenyl acetate was achieved.

FIG. 1H. Combined growth and isoprenyl acetate production of strains.

FIG. 2. Diagram of the IPP-bypass pathway depicting acetylation of isoprenol to isoprenyl acetate, the mevalonate (MVA) pathway, and production of the isoprenoid precursors IPP and DMAPP.

FIG. 3. Overall pathway of DMCO production from glucose. A proposed chemical synthesis converts isoprenyl acetate to isoprene, then oxidative cyclization of two isoprene molecules yields 1,6-DMCOD, which can readily undergo reductive elimination to DMCO. The latter conversion of isoprene to DMCO.

FIG. 4. Amino acid sequence comparison between Saccharomyces cerevisiae PMD (Seq_1, SEQ ID NO:1) and S. epidermis PMD (Seq_2, SEQ ID NO:2).

FIG. 5. Comparison of the amino acid sequence of the ATF1 and ATF2 genes of Saccharomyces cerevisiae (SEQ ID NO:4 and SEQ ID NO:5, respectively), and the Lg-ATF1 gene of Saccharomyces carlsbergensis (SEQ ID NO:6). (Figure taken from FIG. 3 of Nagasawa, et al., “Cloning and Nucleotide Sequence of the Alcohol Acetyltransferase II Gene (ATF2) from Saccharomyces cerevisiae Kyokai No. 7,” Biosci. Biotechnol. Biochem. 62(10): 1852-1857, 1998; herein incorporated by reference).

FIG. 6. Isoprenyl acetate production by JBEI-137161 and variants over 72 hours (JBEI-3606 and acs harboring strains) in M9-MOPS medium with 5 g/L yeast extract.

FIG. 7. Isoprenol production by JBEI-137161 and variants over 72 hours (JBEI-3606 and acs harboring strains) in M9-MOPS medium with 5 g/L yeast extract.

FIG. 8. Isoprenyl acetate production by JBEI-137162 and variants over 72 hours (JBEI-3606 and acs harboring strains) in M9-MOPS medium with 5 g/L yeast extract.

FIG. 9. Isoprenol production by JBEI-137162 and variants over 72 hours (JBEI-3606 and acs harboring strains) in M9-MOPS medium with 5 g/L yeast extract.

FIG. 10. Isoprenyl acetate production by JBEI-137162 and variants over 72 hours (JBEI-3606 and acs harboring strains) in M9-MOPS medium with 5 g/L yeast extract.

FIG. 11. Isoprenol production by JBEI-137162 and variants over 72 hours (JBEI-3606 and acs harboring strains) in M9-MOPS medium with 5 g/L yeast extract.

FIG. 12. Acetate and glucose in isoprenyl acetate production cultures after 72 hours.

FIG. 13. Isoprenyl acetate titer by production cultures after 72 hours in M9-MOPS medium without yeast extract.

FIG. 14. Remaining isoprenyl acetate in 5 mL M9-MOPS cultures after 72 hours of incubation at 30° C. in unsealed culture tubes without cells represented as concentration remaining.

FIG. 15. Endpoint isoprenyl acetate in 5 mL M9-MOPS cultures after 72 hours of incubation at 30° C. in unsealed culture tubes without cells represented as a fraction.

FIG. 16. E. coli DH1 growth in the presence of various concentrations of isoprenyl acetate at inoculation with and without oleyl alcohol overlay. OD600 is normalized to their respective control at 0 g/L isoprenyl acetate.

FIG. 17. Annotated ¹H NMR spectrum of purified chemically synthesized isoprenyl acetate.

FIG. 18A. Isoprenyl acetate production by continuous off-gas capture (A) no overlay.

FIG. 18B. Isoprenyl acetate production by continuous off-gas capture (B) 5% overlay.

FIG. 18C. Isoprenyl acetate production by continuous off-gas capture (C) 20% overlay.

### DETAILED DESCRIPTION OF THE INVENTION

Before the invention is described in detail, it is to be understood that, unless otherwise indicated, this invention is not limited to particular sequences, expression vectors, enzymes, host cells, microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

The terms “host cell” and “host microorganism” are used interchangeably herein to refer to a living biological cell, such as a microorganism, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous DNA” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids comprises two or more subsequences that are not found in the same relationship to each other in nature. The term “heterologous” as used herein refers to a structure or molecule wherein at least one of the following is true: (a) the structure or molecule is foreign to (i.e., not naturally found in) a given host cell; or (b) the structure or molecule may be naturally found in a given host cell, but in an unnatural (e.g., greater than expected) amount. For example, regarding instance (c), a heterologous nucleic acid sequence that is recombinantly produced will have two or more sequences from unrelated genes arranged to make a new functional nucleic acid. Specifically, the present invention describes the introduction of an expression vector into a host cell, wherein the expression vector contains a nucleic acid sequence coding for an enzyme that is not normally found in a host cell. With reference to the host cell's genome, then, the nucleic acid sequence that codes for the enzyme is heterologous.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host cell or cell. Only when the sequence of nucleic acids becomes stably replicated by the cell does the host cell or cell become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host cell, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, the terms “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; internucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

The term “operably linked” refers to a functional linkage between a nucleic acid expression control sequence (such as a promoter) and a second nucleic acid sequence, wherein the expression control sequence directs transcription of the nucleic acid corresponding to the second sequence.

In some embodiments, the genetically modified host cell further comprises an increased expression of one or more of AtoB, hydroxymethylglutaryl-CoA synthase (HMGS), hydroxymethylglutaryl-CoA reductase (HMGR), and/or MK, or a homologous enzyme thereof.

In some embodiments, one or more of the described expressed enzymes, such as PMD, ATF1, AtoB, HMGS, HMGR, and/or MK, or a homologous enzyme thereof, are encoded on one or more nucleotide sequences which are in one or more nucleic acids which are transformed into the genetically modified host cell, or host cell prior to genetic modification. In some embodiments, the nucleotide sequences encoding the one or more enzymes are operatively linked to one or more promoters capable of transcription in the genetically modified host cell. In some embodiments, each nucleic acid of the one or more nucleic acids is a vector capable of stable introduction into and/or maintenance in the host cell.

In some embodiment, the culturing step is culturing the genetically modified host cell under a condition wherein PMD and/or ATF1 are expressed. In some embodiments, the culturing step further comprises expressing AtoB, HMGS, HMGR, and/or MK. In some embodiments, the culturing step is under an anaerobic or microaerobic condition.

In some embodiments, one or more of the enzymes, including PMD, ATF1, AtoB, HMGS, HMGR, and MK, is an engineered enzyme, or homologous, mutant or variant enzymes having the same enzymatic activity, with an amino acid sequence having equal to or more than 70%, 75%, 80%, 85%, 90%, 95%, or 99% identity to the amino acid sequence of the corresponding wild-type enzyme, such as the specific enzymes described in U.S. Pat. Nos. 10,273,506 and 11,001,838 (both of which are incorporated by reference). In some embodiments, one or more of the enzymes, including PMD, ATF1, AtoB, HMGS, HMGR, and MK, is heterologous to the host cell.

In some embodiments, the genetically modified host cell is capable of producing one or more compounds in titers or yields equal to or more than the titers or yields described herein.

In some embodiments, the genetically modified host cell capable of producing an olefinic ester, such as isoprenyl acetate, comprising (a) an increased expression of phosphomevalonate decarboxylase (PMD), or homologous enzyme thereof, wherein the PMD has an amino acid sequence having at least 70% identity with SEQ ID NO:1 or SEQ ID NO:2, and (i) amino acid residue at position 74 is histidine, (ii) amino acid residue at position 145 is phenylalanine, or (iii) amino acid residue at position 74 is histidine and amino acid residue at position 145 is phenylalanine, (b) an increased expression of ATF1, or homologous enzyme thereof, capable of converting isoprenol into isoprenyl acetate, and (c) optionally the genetically modified host cell does not express, or has a decreased expression of one or more of dihydroneopterin triphosphate diphosphate (NudB) and/or phosphomevalonate kinase (PMK); wherein the host cell is a bacterial or fungal cell.

In some embodiments, the decreased expression is a disruption of the promoter or knock out of the gene encoding NudB and/or PMK. In some embodiments, the genetically modified host cell further comprises an increased expression of one or more of acetyl-CoA acetyltransferase (AtoB), hydroxymethylglutaryl-CoA synthase (HMGS), hydroxymethylglutaryl-CoA reductase (HMGR), and/or mevalonate kinase (MK).

In some embodiments, the PMD and/or ATF1 is encoded on a nucleotide sequence which is in nucleic acids which is transformed into the genetically modified host cell, or host cell prior to genetic modification. In some embodiments, one or more of the PMD, ATF1, AtoB, HMGS, HMGR, and MK, are encoded on one or more nucleotide sequences which are in one or more nucleic acids which are transformed into the genetically modified host cell, or host cell prior to genetic modification.

In some embodiments, the genetically modified host cell is a host cell or microorganism, disclosed by U.S. Pat. Nos. 9,752,163; 9,879,286; 10,273,506; and, 10,814,724; and, PCT International Patent Application Nos. PCT/EP2009/067784 and PCT/US2021/040757; and by Zheng, et al. “Metabolic engineering of Escherichia coli for high-specificity production of isoprenol and prenol as next generation of biofuels,” Biotechnology for Biofuels 6:57 (2013); George, et al. “Metabolic engineering for the high-yield production of isoprenoid-based C₅ alcohols in E. coli,” Scientific Reports 5:11128 (2015); Wang, et al. “Tolerance Characterization and Isoprenol Production of Adapted Escherichia coli in the Presence of Ionic Liquids,” ACS Sustainable Chem. Eng. 7(1):1457-1463 (2018); and, Kim et al. “S production,” Metabolic Engineering 64:154-166 (2021) (whereby all are incorporated by reference in their entireties).

In some embodiments, the PMD comprises the following amino acid residues: (a) E at position 73, S at position 108, N at position 110, A at position 119, S at position 120, S at position 121, A at position 122, S at position 155, R at position 158, S at position 208, and/or D at position 302 corresponding to SEQ ID NO:1; or (b) E at position 71, S at position 94, N at position 96, A at position 105, S at position 106, S at position 107, A at position 108, S at position 141, R at position 144, S at position 192, and/or D at position 283 corresponding to SEQ ID NO:2.

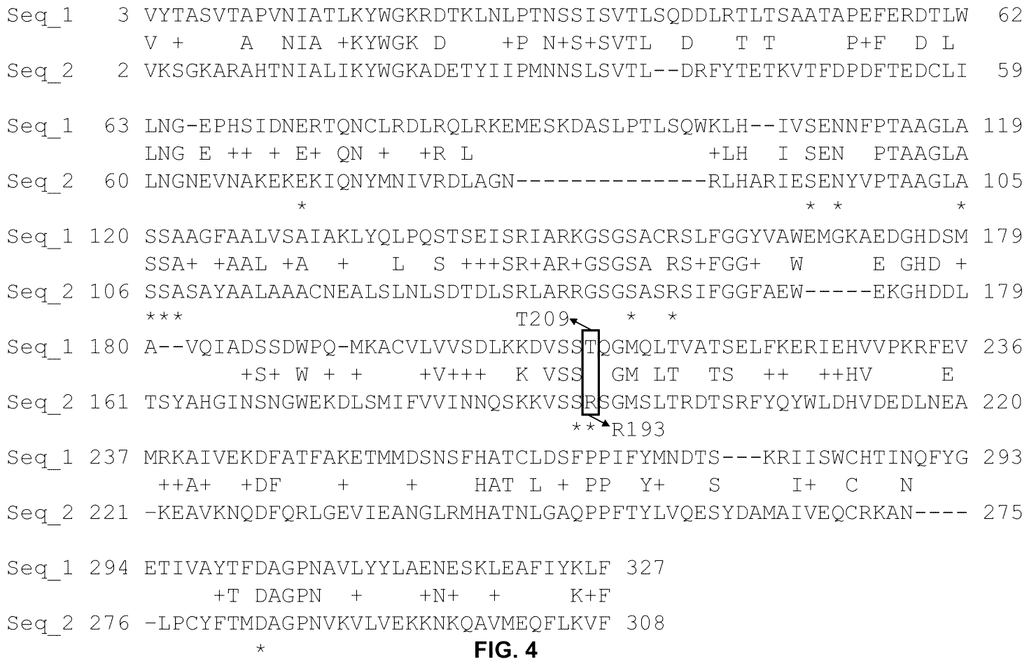

The PMD is any suitable PMD, such as any PMD with an amino acid sequence substantially identical to the amino acid sequences of SEQ ID NO: 1 or 2. The substantially identical PMD comprises one or more, or all, of the conserved residues are identified in FIG. 4, including but not limited to one or more, or all, of the conserved residues indicated by a star.

The amino acid sequence of Saccharomyces cerevisiae PMD (SEQ ID NO:1) is as follows:

```
                10 20 30 40
MTVYTASVTA PVNIATLKYW GKRDTKLNLP TNSSISVTLS
                50 60 70 80
QDDLRTLTSA ATAPEFERDT LWLNGEPHSI DNERTQNCLR
                90 100 110 120
DLRQLRKEME SKDASLPTLS QWKLHIVSEN NEPTAAGLAS
              130 140 150 160
SAAGFAALVS AIAKLYQLPQ STSEISRIAR KGSGSACRSL
              170 180 190 200
FGGYVAWEMG KAEDGHDSMA VQIADSSDWP QMKACVLVVS
              210 220 230 240
DIKKDVSSTQ GMQLTVATSE LFKERIEHVV PKRFEVMRKA
              250 260 270 280
IVEKDFATFA KETMMDSNSF HATCLDSFPP IFYMNDTSKR
              290 300 310 320
IISWCHTINQ FYGETIVAYT FDAGPNAVLY YLAENESKLF
              330 340 350 360
AFIYKLFGSV PGWDKKFTTE QLEAFNHQFE SSNFTARELD
              370 380 390
LELQKDVARV ILTQVGSGPQ ETNESLIDAK TGLPKE
```

The amino acid sequence of Saccharomyces epidermis PMD (SEQ ID NO:2) is as follows:

```
Met Val Lys Ser Gly Lys Ala Arg Ala His Thr Asn Ile Ala Leu Ile
1 5 10 15
Lys Tyr Trp Gly Lys Ala Asp Glu Thr Tyr Ile Ile Pro Met Asn Asn
                        20 25 30
Ser Leu Ser Val Thr Leu Asp Arg Phe Tyr Thr Glu Thr Lys Val Thr
                35 40 45
Phe Asp Pro Asp Phe Thr Glu Asp Cys Leu Ile Leu Asn Gly Asn Glu
        50 55 60
Val Asn Ala Lys Glu Lys Glu Lys Ile Gln Asn Tyr Met Asn Ile Val
65 70 75 80
Arg Asp Leu Ala Gly Asn Arg Leu His Ala Arg Ile Glu Ser Glu Asn
                                85 90 95
Tyr Val Pro Thr Ala Ala Gly Leu Ala Ser Ser Ala Ser Ala Tyr Ala
                        100 105 110
Ala Leu Ala Ala Ala Cys Asn Glu Ala Leu Ser Leu Asn Leu Ser Asp
                115 120 125
Thr Asp Leu Ser Arg Leu Ala Arg Arg Gly Ser Gly Ser Ala Ser Arg
        130 135 140
Ser Ile Phe Gly Gly Phe Ala Glu Trp Glu Lys Gly His Asp Asp Leu
145 150 155 160
Thr Ser Tyr Ala His Gly Ile Asn Ser Asn Gly Trp Glu Lys Asp Leu
                                165 170 175
Ser Met Ile Phe Val Val Ile Asn Asn Gln Ser Lys Lys Val Ser Ser
                        180 185 190
Arg Ser Gly Met Ser Leu Thr Arg Asp Thr Ser Arg Phe Tyr Gln Tyr
                195 200 205
Trp Leu Asp His Val Asp Glu Asp Leu Asn Glu Ala Lys Glu Ala Val
        210 215 220
Lys Asn Gln Asp Phe Gln Arg Leu Gly Glu Val Ile Glu Ala Asn Gly
225 230 235 240
Leu Arg Met His Ala Thr Asn Leu Gly Ala Gln Pro Pro Phe Thr Tyr
                                245 250 255
Leu Val Gln Glu Ser Tyr Asp Ala Met Ala Ile Val Glu Gln Cys Arg
                        260 265 270
Lys Ala Asn Leu Pro Cys Tyr Phe Thr Met Asp Ala Gly Pro Asn Val
                275 280 285
Lys Val Leu Val Glu Lys Lys Asn Lys Gln Ala Val Met Glu Gln Phe
        290 295 300
Leu Lys Val Phe Asp Glu Ser Lys Ile Ile Ala Ser Asp Ile Ile Ser
305 310 315 320
Ser Gly Val Glu Ile Ile Lys
                                325
```

The amino acid sequence of Saccharomyces cerevisiae ATF1 (SEQ ID NO:3) is as follows:

```
                10 20 30 40
MNEIDGKSQG PVQQECLKEM IQNRHARCMG SVEDLYVALN
                50 60 70 80
REHLYRNFCT YGELSDYCSR DQLTLALREI CLKNPTLLHI
                90 100 110 120
VLPTRWPNHE NYYRSSEYYS RPHPVHDYIS VLQELKLSGV
              130 140 150 160
VLNEQPEYSA VMKQILEEFK NSEGSYNAGV FKLSTTLTIP
              170 180 190 200
YFGPTGPSWR LICLPEEHTE KWKKFIFVSN HCMSDGRSSI
              210 220 230 240
HEFHDLRDEL NNIKTPPKKL DYIFKYEEDY QLLRKLPEPI
              250 260 270 280
EKVIDFRPPY LFIPKSLLSG FIYNHLRFSS KGVCMRMDDV
              290 300 310 320
EKTDDVVTEI INISPTEFQA IKANIKSNIQ GKCTITPFLK
              330 340 350 360
GCWFVSLHKW GKFFKPLNFE WLTDIFIPAD CRSQLPDDDE
              370 380 390 400
MRQMYRYGAN VGFIDFTPWI SEFDMNDNKE NFWPLIEHYH
              410 420 430 440
EVISEALRNK KHLHGLGFNI QGFVQKYVNI DKVMCDRAIG
              450 460 470 480
KRRGGTLLSN VGLFNQLEEP DAKYSICDLA FGQFQGSWHQ
              490 500 510 520
AFSLGVCSTN VKGMNIVVAS TKNVVGSQES LEENFEWLTV I
```

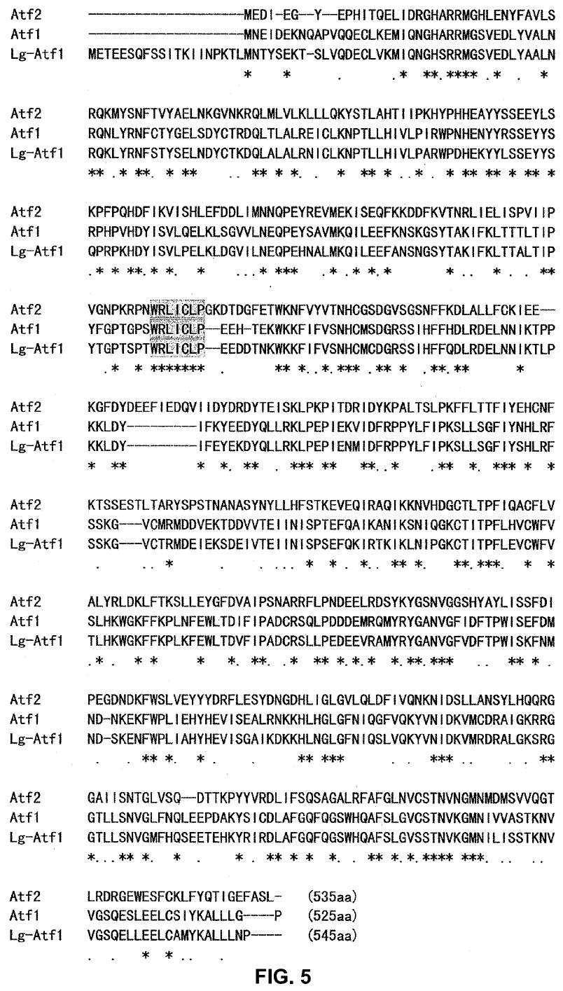

In some embodiments, the ATF1 comprises one or more of the conserved amino acid residues indicated by an asterisk and/or period shown in FIG. 5.

The amino acid sequence of E. coli ACS (SEQ ID NO:7) is as follows:

```
                10 20 30 40
MSQIHKHTIP ANIADRCLIN PQQYEAMYQQ SINVPDTFWG
                50 60 70 80
EQGKILDWIK PYQKVKNTSF APGNVSIKWY EDGTLNLAAN
                90 100 110 120
CLDRHLQENG DRTAIIWEGD DASQSKHISY KELHRDVCRF
              130 140 150 160
ANTLLELGIK KGDVVAIYMP MVPEAAVAML ACARIGAVHS
              170 180 190 200
VIFGGFSPEA VAGRIIDSNS RLVITSDEGV RAGRSIPLKK
              210 220 230 240
NVDDALKNPN VTSVEHVVVL KRTGGKIDWQ EGRDLWWHDL
              250 260 270 280
VEQASDQHQA EEMNAEDPLF ILYTSGSTGK PKGVLHTTGG
              290 300 310 320
YLVYAALTFK YVFDYHPGDI YWCTADVGWV TGHSYLLYGP
              330 340 350 360
LACGATTLMF EGVPNWPTPA RMAQVVDKHQ VNILYTAPTA
              370 380 390 400
IRALMAEGDK AIEGTDRSSL RILGSVGEPI NPEAWEWYWK
              410 420 430 440
KIGNEKCPVV DTWWQTETGG FMITPLPGAT ELKAGSATRP
              450 460 470 480
FFGVQPALVD NEGNPLEGAT EGSLVITDSW PGQARTLFGD
              490 500 510 520
HERFEQTYFS TFKNMYFSGD GARRDEDGYY WITGRVDDVL
              530 540 550 560
NVSGHRLGTA EIESALVAHP KIAEAAVVGI PHNIKGQAIY
              570 580 590 600
AYVTLNHGEE PSPELYAEVR NWVRKEIGPL ATPDVLHWTD
              610 620 630 640
SLPKTRSGKI MRRILRKIAA GDTSNLGDTS TLADPGVVEK
              650
LLEEKQAIAM PS
```

The amino acid sequence of Salmonella enterica ACS (SEQ ID NO:8) is as follows:

```
                10 20 30 40
MSQTHKHAIP ANIADRCLIN PEQYETKYKQ SINDPDTFWG
                50 60 70 80
EQGKILDWIT PYQKVKNTSF APGNVSIKWY EDGTLNLAAN
                90 100 110 120
CLDRHLQENG DRTAIIWEGD DTSQSKHISY RELHRDVCRF
              130 140 150 160
ANTLLDLGIK KGDVVAIYMP MVPEAAVAML ACARIGAVHS
              170 180 190 200
VIFGGFSPEA VAGRIIDSSS RLVITADEGV RAGRSIPLKK
              210 220 230 240
NVDDALKNPN VTSVEHVIVL KRIGSDIDWQ EGRDLWWRDL
              250 260 270 280
IEKASPEHQP EAMNAEDPLF ILYTSGSTGK PKGVLHTTGG
              290 300 310 320
YLVYAATTFK YVFDYHPGDI YWCTADVGWV TGHSYLLYGP
              330 340 350 360
LACGATTLMF EGVPNWPTPA RMCQVVDKHQ VNILYTAPTA
              370 380 390 400
IRALMAEGDK AIEGTDRSSL RILGSVGEPI NPEAWEWYWK
              410 420 430 440
KIGKEKCPVV DTWWQTETGG FMITPLPGAI ELKAGSATRP
              450 460 470 480
FFGVQPALVD NEGHPQEGAT EGNLVITDSW PGQARTLFGD
              490 500 510 520
HERFEQTYFS TFKNMYFSGD GARRDEDGYY WITGRVDDVL
              530 540 550 560
NVSGHRLGTA EIESALVAHP KIAEAAVVGI PHAIKGQAIY
              570 580 590 600
AYVTLNHGEE PSPELYAEVR NWVRKEIGPL ATPDVLHWTD
              610 620 630 640
SLPKTRSGKI MRRILRKIAA GDTSNLGDTS TLADPGVVEK
              650
LLEEKQAIAM PS
```

The amino acid sequence of Salmonella enterica ACS having a substitution mutation of Leucine at position 641 with a Proline (SEQ ID NO:9) is as follows:

(SEQ ID NO: 9)
MSQTHKHAIPANIADRCLINPEQYETKYKQSINDPDTFWGEQGKILDWITPYQKVKNTSFAP
GNVSIKWYEDGTLNLAANCLDRHLQENGDRTAIIWEGDDTSQSKHISYRELHRDVCRFANTL
LDLGIKKGDVVAIYMPMVPEAAVAMLACARIGAVHSVIFGGFSPEAVAGRIIDSSSRLVITA
DEGVRAGRSIPLKKNVDDALKNPNVTSVEHVIVLKRTGSDIDWQEGRDLWWRDLIEKASPEH
QPEAMNAEDPLFILYTSGSTGKPKGVLHTTGGYLVYAATTFKYVFDYHPGDIYWCTADVGWV
TGHSYLLYGPLACGATTLMFEGVPNWPTPARMCQVVDKHQVNILYTAPTAIRALMAEGDKAI
EGTDRSSLRILGSVGEPINPEAWEWYWKKIGKEKCPVVDTWWQTETGGFMITPLPGAIELKA
GSATRPFFGVQPALVDNEGHPQEGATEGNLVITDSWPGQARTLFGDHERFEQTYFSTFKNMY
FSGDGARRDEDGYYWITGRVDDVLNVSGHRLGTAEIESALVAHPKIAEAAVVGIPHAIKGQA
IYAYVTLNHGEEPSPELYAEVRNWVRKEIGPLATPDVLHWTDSLPKTRSGKIMRRILRKIAA
GDTSNLGDTSTLADPGVVEKPLEEKQAIAMPS

### Enzymes, and Nucleic Acids Encoding Thereof

A homologous enzyme is an enzyme that has a polypeptide sequence that is at least 70%, 75%, 80%, 85%, 90%, 95% or 99% identical to any one of the enzymes described in this specification or in an incorporated reference. The homologous enzyme retains amino acids residues that are recognized as conserved for the enzyme. The homologous enzyme may have non-conserved amino acid residues replaced or found to be of a different amino acid, or amino acid(s) inserted or deleted, but which does not affect or has insignificant effect on the enzymatic activity of the homologous enzyme. The homologous enzyme has an enzymatic activity that is identical or essentially identical to the enzymatic activity any one of the enzymes described in this specification or in an incorporated reference. The homologous enzyme may be found in nature or be an engineered mutant thereof.

The nucleic acid constructs of the present invention comprise nucleic acid sequences encoding one or more of the subject enzymes. The nucleic acid of the subject enzymes is operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, Xhol, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host cell simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and X phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host cell. Also, microinjection of the nucleic acid sequencers) provides the ability to transfect host cell. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

When the host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of the isoprenol ensured. When added, any intermediate is present in an excess amount in the culture medium.

The present invention provides for a method for constructing genetically modified host cell of the present invention comprising: (a) introducing one or more nucleic acid comprising open reading frames (ORF) encoding the enzymes described herein wherein each is operatively linked to a promoter capable of transcribing each ORF to which it is operatively linked, and/or (b) optionally knocking out one or more of the enzymes described herein such that the modified host cell does not express the one or more knocked out enzymes.

### Host Cells

The genetically modified host cell can be any prokaryotic or eukaryotic cell, with any genetic modifications, capable of production of the olefinic ester in accordance with the methods of the invention. Suitable eukaryotic host cells include, but are not limited to, fungal cells. Suitable fungal cells are yeast cells, such as yeast cells of the Saccharomyces genus. Generally, although not necessarily, the host cell is a yeast or a bacterium. Any prokaryotic or eukaryotic host cell may be used in the present method so long as it remains viable after being transformed with a sequence of nucleic acids. In some embodiments, the host cell is not adversely affected by the transduction of the necessary nucleic acid sequences, the subsequent expression of the proteins (i.e., enzymes), or the resulting intermediates required for carrying out the steps associated with the mevalonate pathway. For example, it is preferred that minimal “cross-talk” (i.e., interference) occur between the host cell's own metabolic processes and those processes involved with the mevalonate pathway.

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

The enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments, the host cell is Rhodosporidium toruloides or Pseudomonas putida. In some embodiments, the host cell is a Gram negative bacterium. In some embodiments, the host cell is of the phylum Proteobactera. In some embodiments, the host cell is of the class Gammaproteobacteria. In some embodiments, the host cell is of the order Enterobacteriales. In some embodiments, the host cell is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes.

Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteiii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

Converting Isoprenyl Acetate into Isoprene

Isoprenyl acetate can be converted into isoprene through the following: (i) deacetylating the isoprenyl acetate into isoprene, and/or (ii) hydrolyzing isoprenyl acetate into isoprenol and dehydrating the isoprenol into isoprene.

In some embodiments, the step (c) (i) deacetylating the isoprenyl acetate into isoprene, and/or (ii) hydrolyzing isoprenyl acetate into isoprenol and dehydrating the isoprenol into isoprene comprises: (i) heating an isoprenol fraction to a temperature equal to or more than about 70° C., 75° C., 80° C., 85° C., 90° C., 95° C., or 100° C., or to a temperature within a range between any two of the preceding temperatures, wherein isoprenyl acetate is converted into isoprene, wherein at least some of the isoprene is in a gaseous state; and (ii) condensing or distilling the isoprene in the gaseous state isoprenol. Isoprenyl acetate is hydrolyzed into isoprenol and acetic acid. In some embodiments, the (c) step, (i) deacetylating the isoprenyl acetate into isoprene, and/or (ii) hydrolyzing isoprenyl acetate into isoprenol and dehydrating the isoprenol into isoprene, comprises using a distillation column, and an overhead temperature of the distillation column is decreased from about 50° C., 45° C., 40° C., 35° C., 34° C., 33° C., or 30° C., to about 25° C., 23° C., 22° C., 21° C., 20° C., 15° C., or 10° C., wherein the isoprene in the gaseous state condenses or distills to form a condensate or distillate. In some embodiments, a condensate or distillate obtained from the condensing or distilling step comprises: equal to or more than about 50%, 55%, 60%, 65%, 70%, 75%, 80%, 85%, 90%, or 95% isoprene, less than about 25%, 20%, 15%, 10%, or 5% of isoprenol isomers and isoprenyl acetate isomers, and less than about 1% of dimers. In some embodiments, the distillation column is first charged with an aqueous solution of salt and acid (such as an inorganic acid, such as HCl, H₂SO₄, or HNO₃). In some embodiments, the salt, acid, and water in the aqueous solution have a weight or molar ratio of about 1:0.1:1.5. In some embodiments, the salt is any metal halide, sulfate, or nitrate. In some embodiments, the metal is any alkali, alkaline earth, or transition metal. In some embodiments, the aqueous solution of salt and acid comprises sodium chloride, HCl, and water. (6.01 g). In some embodiments, the aqueous solution is agitated or stirred such as agitated or stirred vigorously. In some embodiments, the agitation or stirring of the aqueous solution takes place at a temperature equal to or more than about 95° C., 90° C., 85° C., 80° C., 75° C., 70° C., 65° C., or 60° C.

The isoprene formed evaporates from the solution. In some embodiments, the isoprenyl acetate in the solution is reduced, or is entirely converted into isoprene, such that the solution is free of isoprenyl acetate, or essentially free of isoprenyl acetate. In both cases the isoprenyl acetate-free water could be returned as a recovery or extraction solvent. In some embodiments, the isoprene is distilled from the metal, degassed and placed under a nitrogen atmosphere. In some embodiments, the aqueous dehydration reaction mixture generates one or more minor byproducts, such as isoprenyl acetate isomers or cyclic isoprene dimer. Such minor byproducts, such as cyclic isoprene dimer, can be readily isolated from the aqueous dehydration reaction mixture and combined with the DMCOD.

Converting Isoprene into 1,4-Dimethylcyclooctane (DMCO)

In some embodiments, the step (e) converting isoprene into DMCO comprises: (i) [4+4]-cyclodimerizing of the isoprene into a 1,6-dimethyl-1,5-cyclooctadiene (DMCOD), and (ii) hydrogenating the DMCOD into a DMCO. In some embodiments, the [4+4]-cyclodimerizing of the isoprene into DMCOD comprises contacting isoprene with a metal catalyst such that the isoprene is converted into DMCOD. In some embodiments, the hydrogenating the DMCOD comprises contacting DMCOD with a metal catalyst such that the DMCOD is converted into DMCO. In some embodiments, the metal catalyst comprises a transition metal, such as Fe and Pt. In some embodiments, the metal catalyst is platinum (IV) oxide.

The cyclic isoprene dimers can be partially dehydrogenated to form one or more C₁₀H₁₄ aromatic compounds, which can be used to further diversify a fuel mixture-aromatic compounds are useful for swelling nitrile rubber elastomers in jet engines. The one or more C₁₀H₁₄ aromatic compounds can be blended with either jet fuel or diesel fuel. Besides the cyclic isoprene dimers, the process can also produce further oligomers, such as a trimer (C15), tetramer (C20), or the like. The C15 oligomer can be hydrogenated and blended with either jet fuel or diesel fuel. The C20 oligomer can be hydrogenated and blended with diesel fuel. Hydrogenated heavier oligomers have utility as lubricants, while unhydrogenated versions can be used as adhesives or hydrophobic sealants. The dehydration process is tolerant of other biologically produced components captured from the fermentation process (such as, isoprenyl acetate). Residual isoprenyl acetate can be isolated from the aqueous reaction mixture and used for other applications (gasoline additive, solvent, or the like) or converted to isoprenol by treatment with base. A C15 trimer and hydrogenated C15 trimer have the following chemical structures:

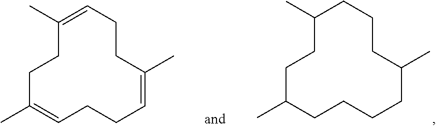

and respectfully.

In some embodiments, the method further comprises: mixing DMCO with a fuel additive to produce a fuel mixture. In some embodiments, the method further comprises: mixing any one, two, three, four, five, six, or all of DMCO, cyclic isoprene dimer, DMCOD, one or more C₁₀H₁₄ aromatic compounds, hydrogenated C15 oligomer, hydrogenated C15 oligomer, with a jet fuel or diesel fuel, and/or with a fuel additive to produce a fuel mixture. In some embodiments, the fuel additive includes one of antioxidants, thermal stability improvers, cetane improvers, stabilizers, cold flow improvers, combustion improvers, anti-foams, anti-haze additives, corrosion inhibitors, lubricity improvers, icing inhibitors, injector cleanliness additives, smoke suppressants, drag reducing additives, metal deactivators, dispersants, detergents, demulsifiers, dyes, markers, static dissipaters, biocides, and combinations thereof.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Production of Isoprenyl Acetate

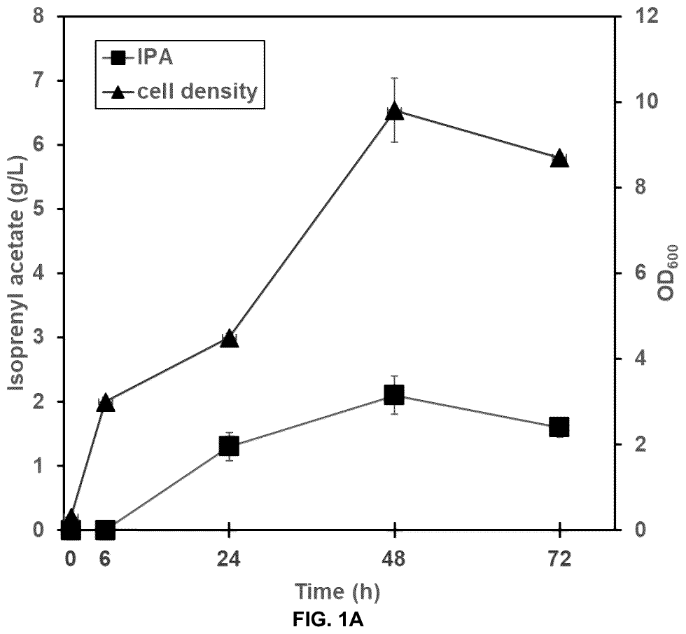

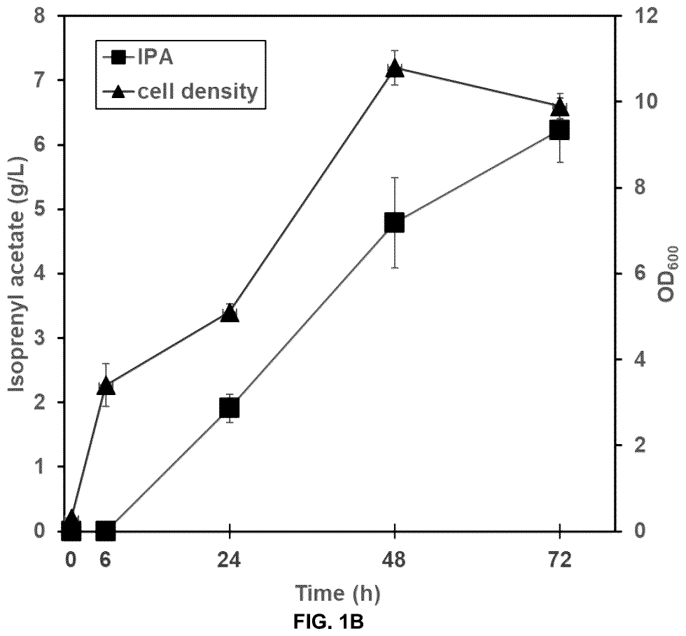

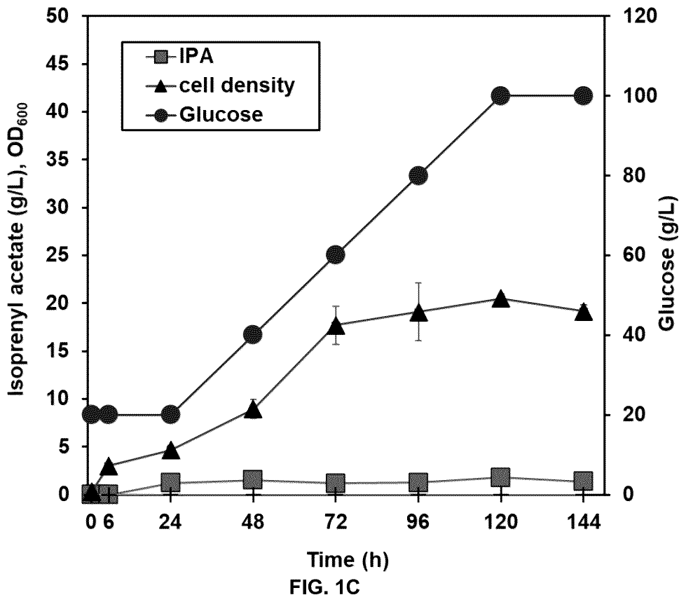

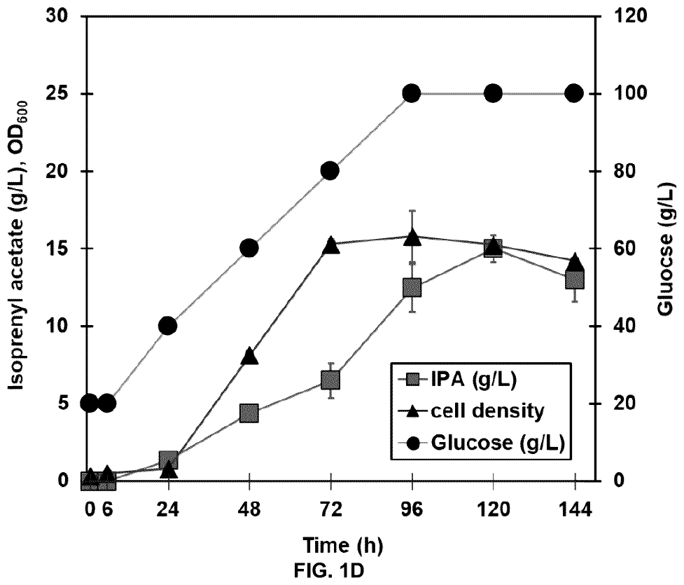

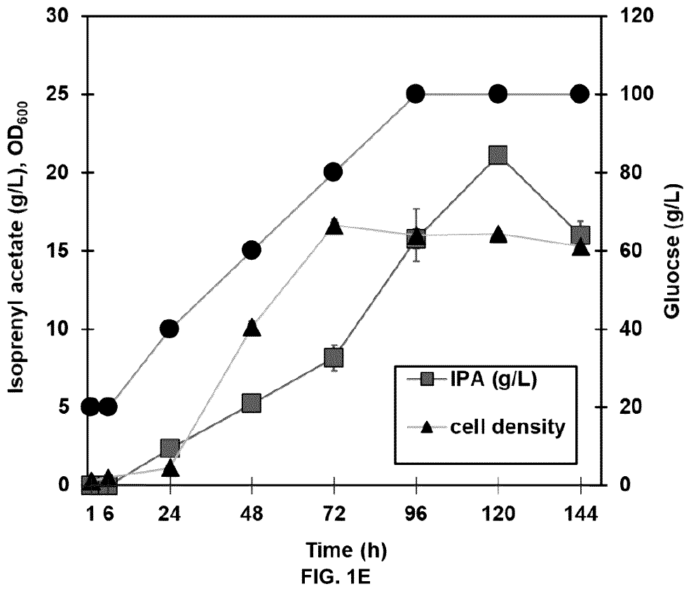

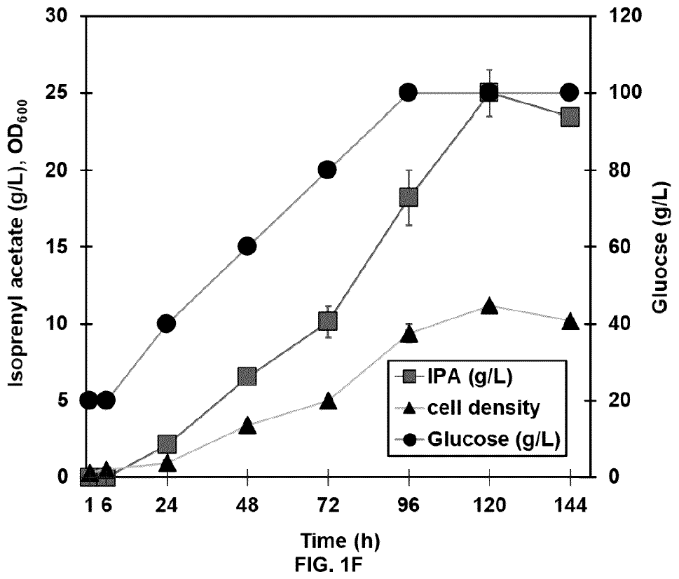

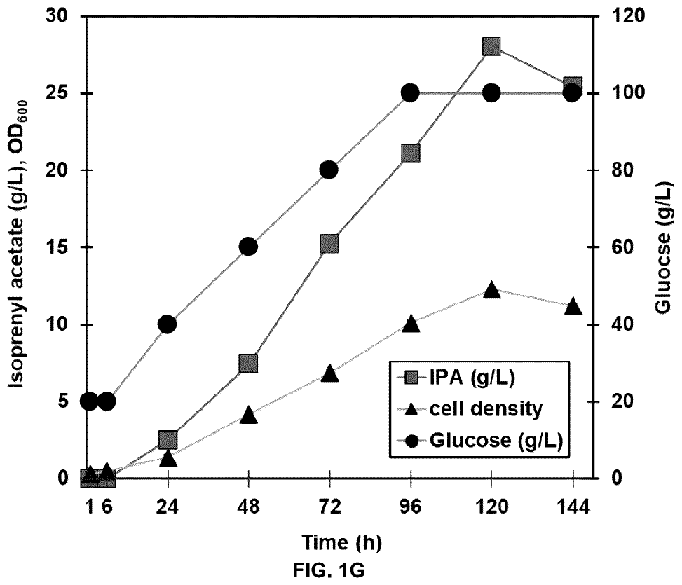

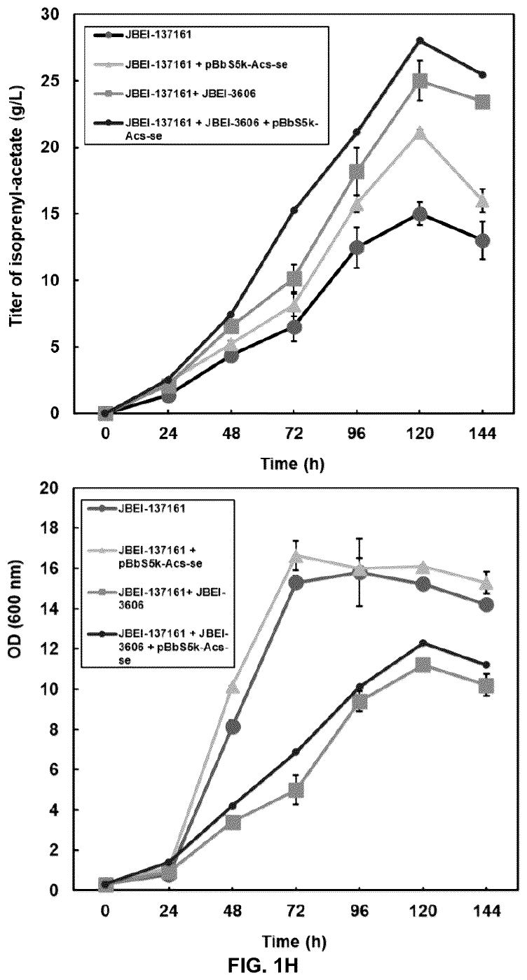

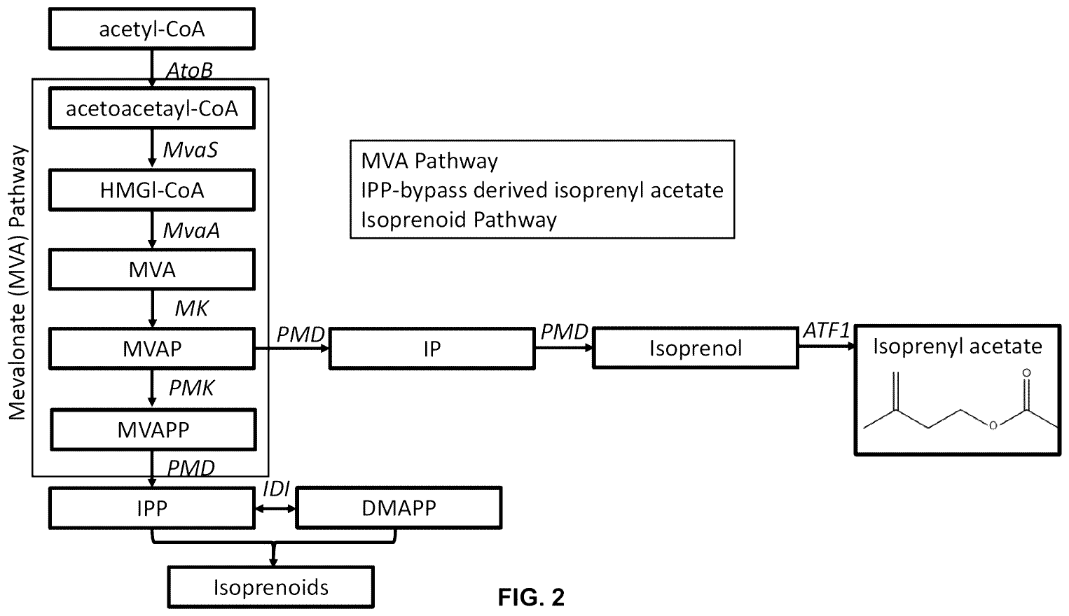

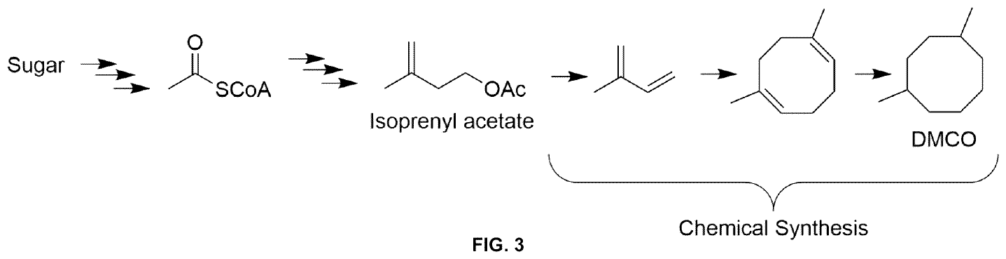

The following describes the initial production of isoprenyl acetate in M9 minimal media with yeast extract under varied fermentation conditions. Initial experiments studied batch fermentation without overlay (FIG. 1A) as well as with an oleyl alcohol overlay (FIG. 1B). These are complimented by fed-batch experiments without overlay (FIG. 1C) and with an oleyl alcohol overlay (FIG. 1D) each showing glucose added, total isoprenyl production, and cell density. FIG. 2 indicates the overall pathway for isoprenyl acetate production from acetyl-CoA via the IPP-bypass within the context of the MVA and isoprenoid pathways. FIG. 3 shows the chemical synthesis of DMCO from isoprenyl acetate precursor.

### Results

Production of Acetate Esters from MVA Derived Alcohols

Microbial ester production pathways consist of three principal components: an alcohol, an acyl-CoA thioester, and an alcohol acyltransferase (AAT) capable of the alcohol acylation. Traditionally, pathways have included a range of yeast and plant AATs with variable selectivity towards a library of common alcohols (ethanol, propanol, and butanol) as well short chain length acyl-CoA thioesters; however, the lack of AAT selectivity often contingent upon substrate availability such that highly selective ester production is a significant challenge. In acylating a primary alcohol like isoprenol, obvious initial acyltransferase candidates included ATF1 and ATF2 from S. cerevisiae, which maintain a noted selectivity towards acetyl-CoA (1) and primary alcohols. A third acyltransferase, SAAT from Fragaria×ananassa, was chosen for its proclivity for longer chain acyl-CoAs. Selected acyltransferases were cloned into the IPP-bypass strain JBEI-137161 in an operon with PMD and MK.

The isoprenyl acetate production pathway collectively requires three acetyl-CoA molecules: two to drive MVA flux and one for the acetylation reaction. As a result, acetyl-CoA and acetate precursor balancing are extremely important to overall production, leading to a significant investigation into acetate pathway knockouts and select gene overexpression. Typically, deletion of pta, poxB, and ackA result in near complete loss of acetate production, which would ideally drive an accumulation of acetyl-CoA. Furthermore, two acetyl-CoA synthetases (ACS) were overexpressed to convert residual acetate into acetyl-CoA substrate. The first acs gene was cloned from E. coli chromosomal DNA (acs_{ec}) while a second was cloned from Salmonella enterica (acsₛₑ). The latter acs harbors a L641P point mutation that reduces acetylation and feedback inhibition, thereby continually converting acetate into acetyl-CoA precursor. Selected acs genes were cloned onto a tertiary, low copy plasmids (pDNC60 and pDNC61) and transformed into production strains.

The FASTA of ACS_{Se}(L641P) with the point mutation bold and underlined in the following (>tr|A0A3U3W6Q2|A0A3U3W6Q2_SALET Acetyl-coenzyme A synthetase OS═Salmonella enterica I OX=59201 GN=acs PE=3 SV=1):

(SEQ ID NO: 9)
MSQTHKHAIPANIADRCLINPEQYETKYKQSINDPDTFWGEQGKILDWITPYQKVKNTSFAP
GNVSIKWYEDGTLNLAANCLDRHLQENGDRTAIIWEGDDTSQSKHISYRELHRDVCRFANTL
LDLGIKKGDVVAIYMPMVPEAAVAMLACARIGAVHSVIFGGFSPEAVAGRIIDSSSRLVITA
DEGVRAGRSIPLKKNVDDALKNPNVTSVEHVIVLKRTGSDIDWQEGRDLWWRDLIEKASPEH
QPEAMNAEDPLFILYTSGSTGKPKGVLHTTGGYLVYAATTFKYVFDYHPGDIYWCTADVGWV
TGHSYLLYGPLACGATTLMFEGVPNWPTPARMCQVVDKHQVNILYTAPTAIRALMAEGDKAI
EGTDRSSLRILGSVGEPINPEAWEWYWKKIGKEKCPVVDTWWQTETGGFMITPLPGAIELKA
GSATRPFFGVQPALVDNEGHPQEGATEGNLVITDSWPGQARTLFGDHERFEQTYFSTFKNMY
FSGDGARRDEDGYYWITGRVDDVLNVSGHRLGTAEIESALVAHPKIAEAAVVGIPHAIKGQA
IYAYVTLNHGEEPSPELYAEVRNWVRKEIGPLATPDVLHWTDSLPKTRSGKIMRRILRKIAA
GDTSNLGDTSTLADPGVVEKPLEEKQAIAMPS

### Isoprenyl Acetate Production in Flask Experiments

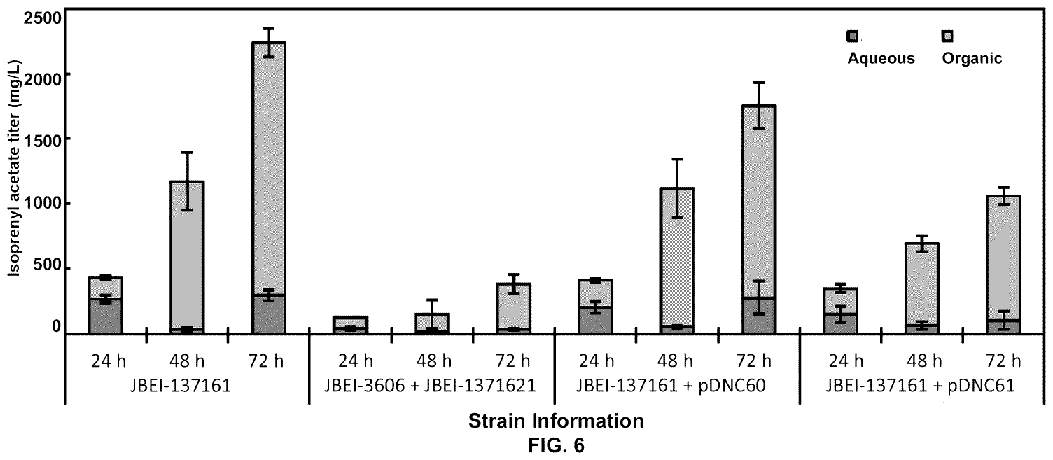

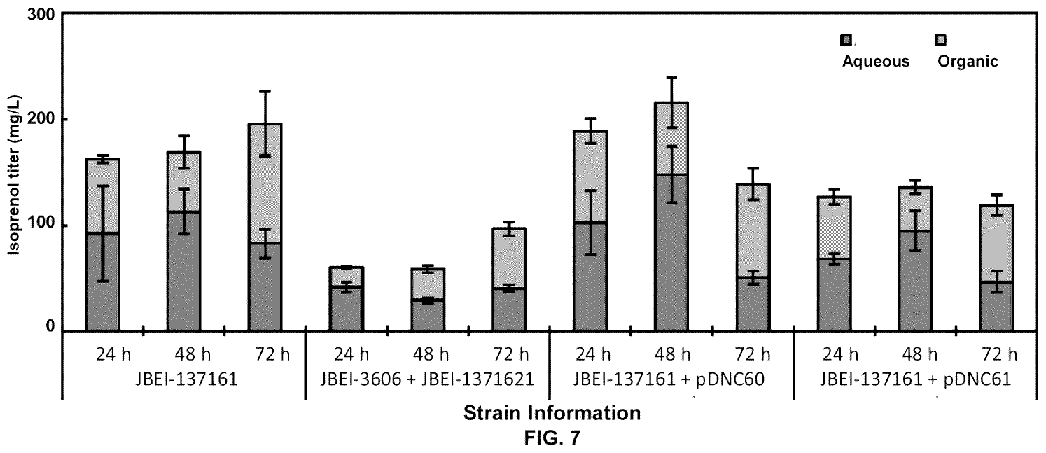

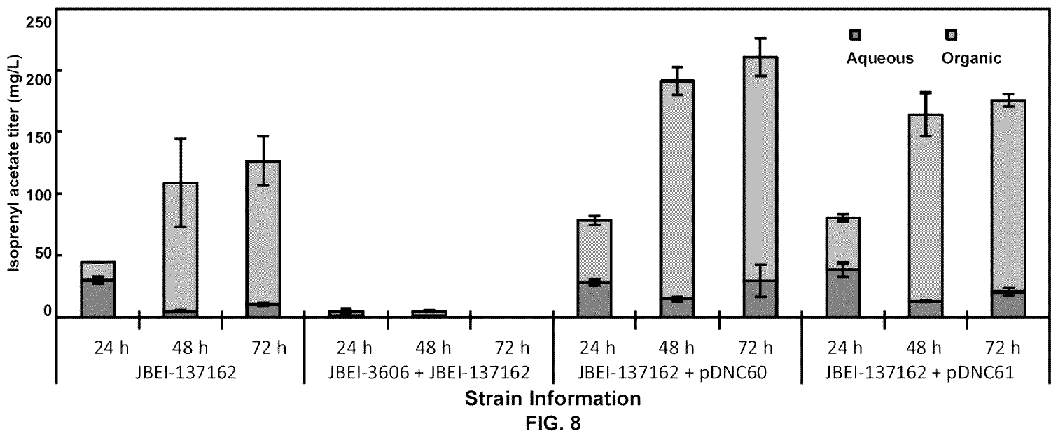

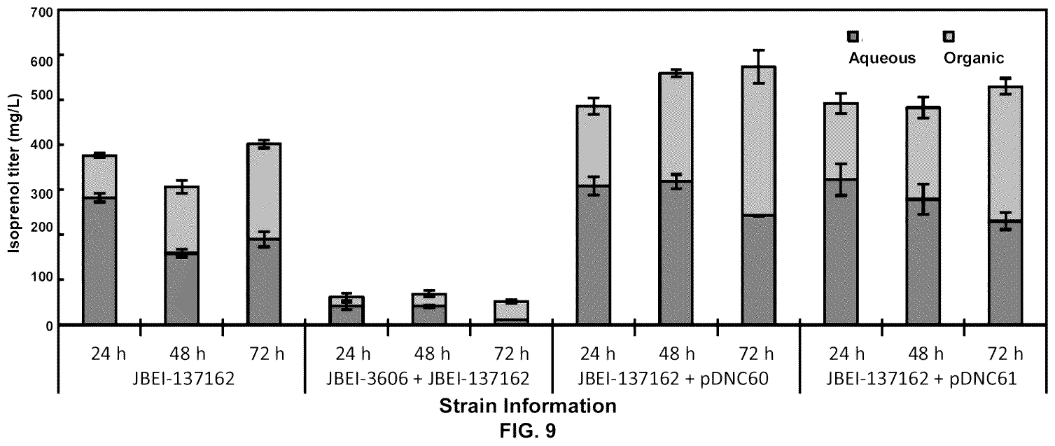

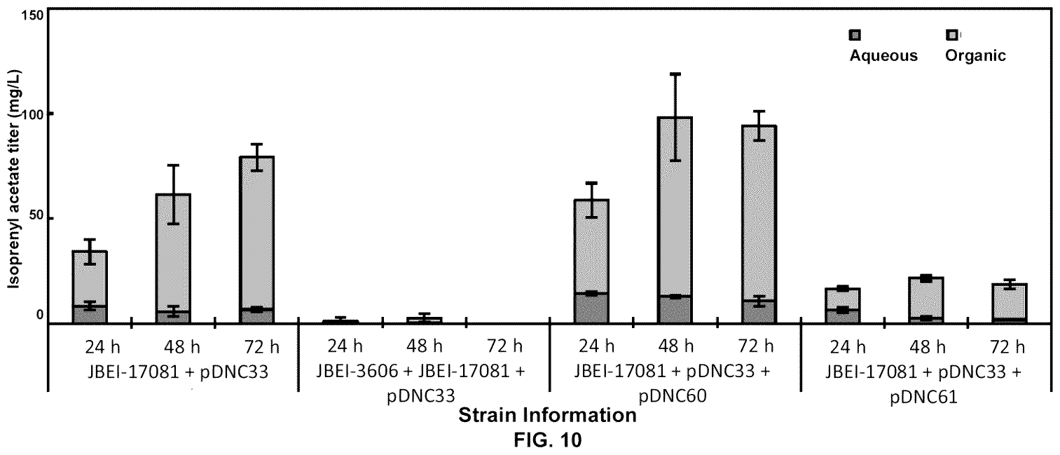

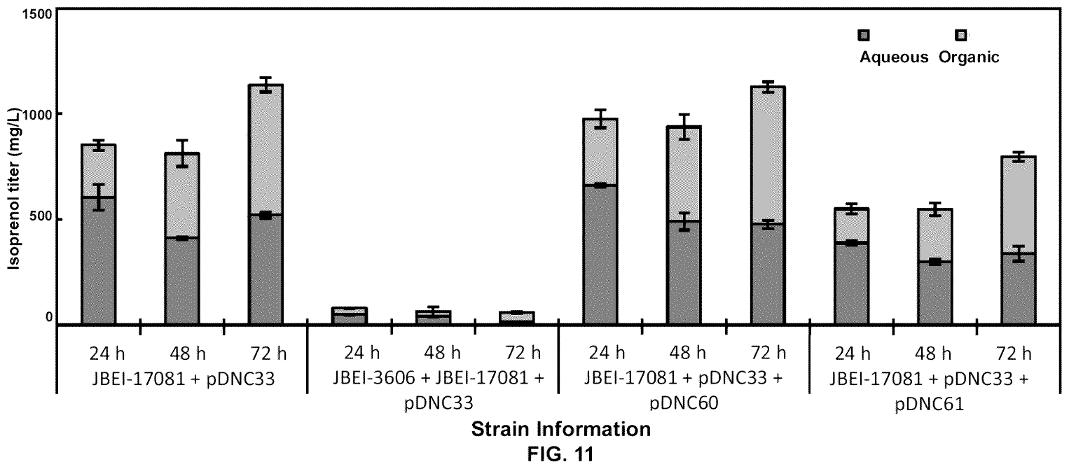

In order to investigate isoprenyl acetate production using heterologous AAT expression, small-scale flask experiments were conducted in M9-MOPS media with yeast extract, the addition of which has previously been employed for high titer isoprenol production. Alcohol, ester, and acetate production from these 12 strains over three days are depicted in FIGS. 6-11.

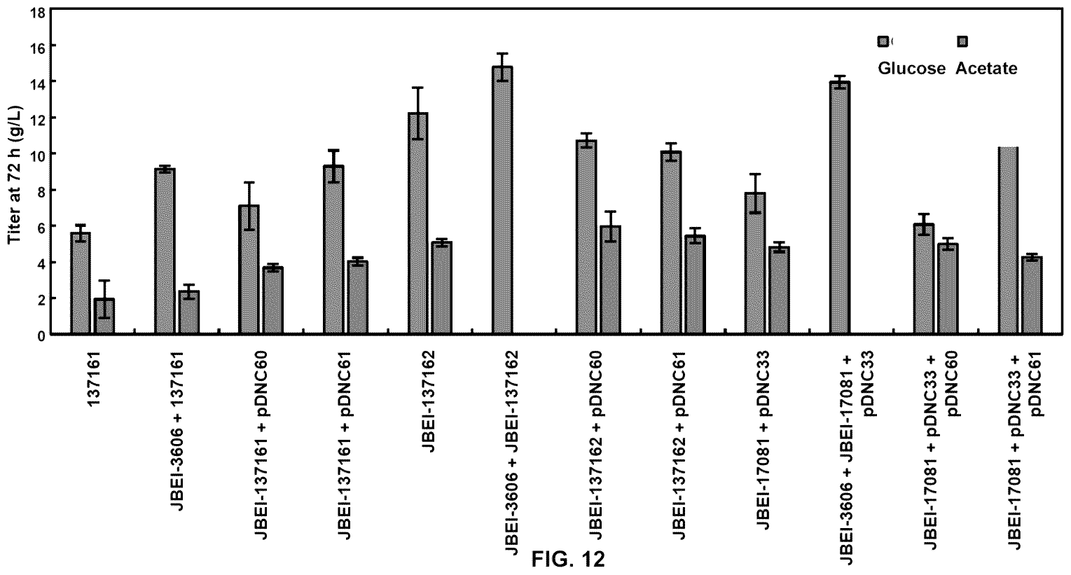

The combination of ATF1 with the IPP-bypass strain demonstrated exceptionally high olefinic ester production, attaining 2.24±0.15 g/L over 72 hours. On the other hand, production by ATF2 and SAAT were limited to maximum titers of 210±28 mg/L and 94±10 mg/L, respectively, with significantly higher accumulation of isoprenol suggesting lack of sufficient selectivity towards either the acetyl-CoA or isoprenol substrates. Interestingly, the presence of an overexpressed acs provided a marked advantage in JBEI-137162 and JBEI-227453, which could indicate improved precursor availability. Yet an analysis of acetate after 72 hours indicated that the overexpression of the acs genes counterintuitively increased acetate accumulation across all AATs (FIG. 12).

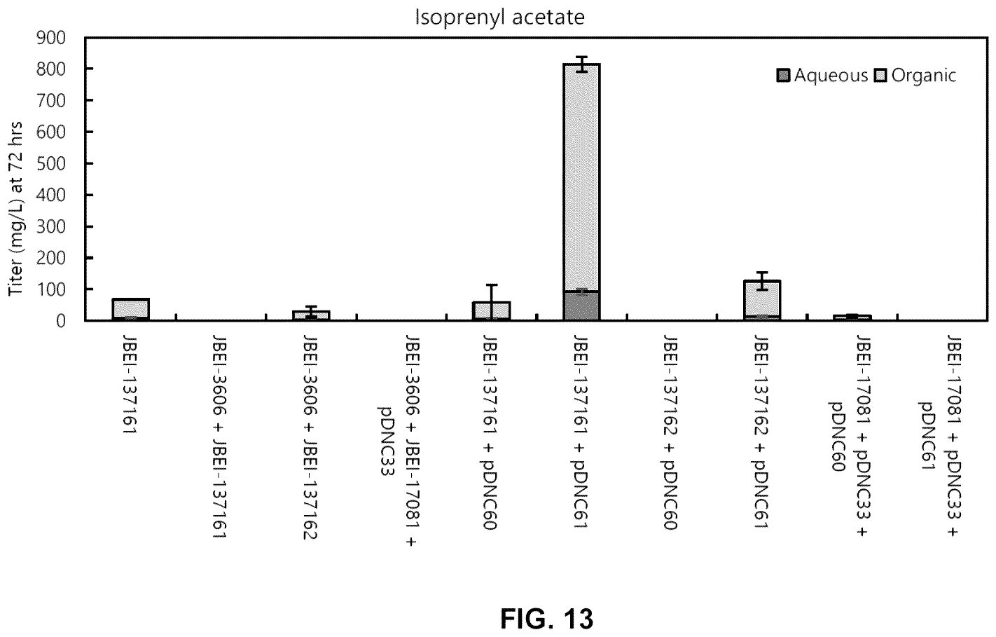

Repeating this experiment in M9-MOPS without yeast extract, however, yielded a significant advantage for isoprenyl acetate production by JBEI-137161 harboring ACSₛₑ due in part to the relative overaccumulation of acetate by 137161 and the ACSₑc (data not shown), a phenomenon only seen in minimal media (FIG. 13). Ostensibly, these data support a nuanced relationship between the media composition, growth conditions, and acetate pathway rebalancing that dictate acetyl-CoA flux and, ultimately, isoprenyl acetate titer. As a result, we proceeded to explore high titer production in a biofermenter in which a high level of control could also better tune acetate accumulation.

### Isoprenyl Acetate Production in Fed-Batch Fermentation

To further improve titer, JBEI-137161 and its variants were used for fed-batch fermentation in a 2 L bioreactor by feeding additional glucose and NH₄Cl as nitrogen source. M9-MOPS minimal medium with 2% glucose was used during the batch fermentation while a feeding solution (80 g/L glucose and 5 g/L NH₄Cl) was continuously added at a constant rate after the initial glucose was consumed. The production of isoprenyl acetate from JBEI-continuously increased during the fermentation, reaching a titer of 15 g/L (0.15 g/g glucose) at 120 hours, (FIG. 1D).

To improve IPA production, the acetyl-CoA supply was promoted by overexpression of acetyl-CoA synthase (ACSₛₑ^{L641P}) which can convert from acetate to acetyl-CoA (2). Fed-batch fermentation of JBEI-227454, which harbors the ACSₛₑ^{L641P} along with the IPP-bypass pathway, resulted in 18 g/L isoprenyl accumulation over 120 hours (FIG. 1E). It was shown that production of isoprenyl acetate was dramatically improved by ACSₛₑ^{L641P} expression, likely due to improving acetyl-CoA availability. However, during the feeding of glucose and nitrogen source, it was observed that a significant amount of acetate was increased after 48 h. The acetate accumulation caused inhibition of growth and low production of target biochemicals in the batch and fed-batch fermentation (3). To reduce the acetate production in fed-batch fermentation, a strain (JBEI-227452) with deletions of select acetate biosynthesis pathway genes (poxB, ackA, and pta) was used for production. Although only a small amount of acetate was detected from the mutant strain (JBEI-227452), there was significantly lower OD_{600 nm} and associated glucose consumption during cultivation (FIG. 1H). Nonetheless, significantly more isoprenyl acetate was produced, reaching 25 g/L after 120 h (FIG. 1F). This result demonstrated that the deletion of acetate biosynthesis prevented the loss of valuable substrate and improved conversion of glucose to ester. It also suggested potentially that these factors were additive. Finally, to improve isoprenyl acetate production, ACSₛₑ^{L641P} was expressed in the mutant strain (JBEI-227476), attaining 28 g/L after 120 hours (FIG. 1G).

### Materials and Methods:

### Construction of Plasmids

All plasmids were constructed by PCR amplifying a target DNA fragment paired with a desired vector harboring an origin and selection marker. All PCRs were performed using NEB Q5 polymerase according to the manufacturer's instructions. In the case of assemblies, PCRs were conducted with oligonucleotides (Integrated DNA Technologies) maintaining ˜20 bp 5′ homology overhangs for scarless integration using NEBuilder HiFi Assembly Master Mix (NEB). Assemblages were then cloned into E. coli XL-1 Blue chemically competent cells for high efficiency plasmid replication. Plasmids harboring the desired sequence were subsequently isolated using a QIAprep Spin Miniprep Kit (Qiagen) and transformed into electrocompetent E. coli DH1 cells via electroporation (2500 V, 5 ms) in 0.2 cm gap cuvettes (Biorad).

To generate acetate esters, two alcohol o-acetyltransferase genes atf1 [NCBI: NP_015022.3] and atf2 [NCBI: NP_011693.1] were amplified directly from S. cerevisiae S288C chromosomal DNA and cloned 3′ of the mevalonate kinase in JBEI-17844, which also harbors a mutagenized PMD enabling IPP-bypass. Another AAT [GenBank: AAG13130.1] from Fragaria×ananassa denoted saat was codon optimized for E. coli and cloned 3′ of the mevalonate kinase of JBEI-17844.

TABLE 1
An abridged list of plasmids and strains used in this
study for the production of isoprenyl acetate.

| Plasmid or | | |
|---|---|---|
| strain | Relevant genotype | Ref |
| JBEI-17081 | pA5c-AtoB-HMGS_Sa-HMGR_Sa | (4) |
| JBEI-17844 | ptrc99a-PMDsc_HKQ-MKmm | (5) |
| JBEI-136483 | ptrc99a-PMDsc_HKQ-MKmm-Atf1 | This work |
| JBEI-136484 | ptrc99a-PMDsc_HKQ-MKmm-Atf2 | This work |
| pDNC33 | ptrc99a-PMDsc_HKQ-MKmm-SAAT | This work |
| pDNC60 | pS5k-ACSec | This work |
| pDNC61 | pS5k-ACSse_L641P | This work |
| DH1 | F⁻ λ⁻ endA1 recA1 relA1 gyrA96 thi-1 glnV44 | Wild type |
| | hsdR17(r_{K}⁻m_{K}⁻) | |
| JBEI-3606 | DH1 Δpta, ΔackA, ΔpoxB | (6) |
| JBEI-137161 | DH1 harboring JBEI-17081, JBEI-136483 | This work |
| JBEI-137162 | DH1 harboring JBEI-17081, JBEI-136484 | This work |
| JBEI-227453 | DH1 harboring JBEI-17081, pDNC33 | This work |
| JBEI-227454 | DH1 harboring JBEI-17081, JBEI-136483, | This work |
| | pDNC60 | |
| JBEI-227452 | DH1 harboring JBEI-17081, JBEI-136483, | This work |
| | pDNC61 | |
| JBEI-227474 | JBEI-3606 harboring JBEI-17081, JBEI- | This work |
| | 136483 | |
| JBEI-227475 | JBEI-3606 harboring JBEI-17081, JBEI- | This work |
| | 136483, pDNC60 | |
| JBEI-227476 | JBEI-3606 harboring JBEI-17081, JBEI- | This work |
| | 136483, pDNC61 | |

### Production Culture Conditions

Seed cultures were initially inoculated from colony into Luria-Bertani (LB) media (10 g/L tryptone, 5 g/L yeast extract, and 10 g/L sodium chloride) for overnight growth at 37° C. on a rotary shaker at 200 rpm. Where applicable, antibiotics included carbenicillin (100 μg/mL), chloramphenicol (34 μg/mL), and kanamycin (50 μg/mL). The production medium was a variation of M9 minimal medium that included M9 salts (6.78 g/L Na₂HPO₄, 3 g/L KH₂PO₄, 1 g/L NH₄Cl, and 0.5 g/L NaCl), 1 mg/L thiamine, 2 mM MgSO₄, 10 nM FeSO₄, 0.1 mM CaCl₂, micronutrients (3*10⁻⁸ M (NH₄)₆Mo₇O₂₄, 4*10⁻⁶ M boric acid, 3*10⁻⁷ M CoCl₂, 1.5*10⁻⁷ M CuSO₄, 8*10⁻⁷ M MnCl₂, and 1*10⁻⁷ M ZnSO₄), as well as 75 mM 3-morpholinopropane-1-sulfonic acid (MOPS), 20 g/L glucose, appropriate antibiotics, and, if stated, 5 g/L yeast extract. Upon overnight growth in LB, seed cultures were inoculated into M9-MOPS media with or without yeast extract for production or adaptation, respectively. Adaptation in M9-MOPS was found to be critical in reducing bacterial lag phase in media without yeast extract.

Flask production cultures were inoculated at an optimal density (λ=600 nm, 1 cm path length) of 0.05 in M9-MOPS using a Spectramax Plus spectrophotometer (Molecular Devices), then incubated at 37° C. with shaking at 200 rpm. Upon reaching an OD600 of 0.4 to 0.6, cultures were induced with 0.5 mM isopropyl 3-D-1-thiogalactopyranoside (IPTG) and transferred to a shaker at 30° C. and 200 rpm. This experimental approach was used for both small-scale 5 mL culture tube and medium-scale 50 mL culture flask experiments. For acetate ester production, a 20% oleyl alcohol overlay was also added to sequester the relatively nonpolar products. Dodecane and hexadecane were also explored as overlay candidates, though oleyl alcohol maintained the best chromatogram clarity with respect to ester and alcohol production. Growth, metabolites, and production were determined daily using OD, high pressure liquid chromatography (HPLC), and gas chromatography-flame ionization detector (GC-FID), respectively.

### Isoprenyl Acetate (IPA) Production in Fed-Batch Fermenter

Fed-batch seed cultures were initially prepared by inoculating 2 mL of M9-MOPS with yeast extract with cells from a glycerol cryostock, then grown for 12 hours. For adaptation to minimal medium, cell cultures were diluted 50-fold into fresh 5 mL M9-MOPS and grown for 24 h. The seed culture was then used to inoculate in 1 L flask with 100 mL M9-MOPS medium, grown at 37 C for 8 h, and then was used to inoculate the bioreactor at OD_{600 nm} of 0.3. Fed-batch fermentations were conducted using a culture volume of 1 L M9-MOPS in 2 L bioreactors (Sartorius BIOSTAT B plus) with isoprenyl acetate biosynthesis induction by addition of 0.5 mM IPTG at an OD₆₀₀ of 0.6. Setpoints for dissolved oxygen (DO), temperature, and airflow were 3%, 30° C., and 1 VVM (volume of air per volume of liquid per minute), respectively. Culture pH was maintained at 6.5 by supplementation with ammonia water (25%).

A feeding solution (80 g/L glucose and 5 g/L NH₄Cl) was delivered by a Watson-Marlow DU520 peristaltic pump at a rate closely matching the batch phase glucose consumption. For exponential feeding, the flow rate was increased every hour over a total of 8 hours and calculated following the Korz's equation (5,7).

$${m}_{s} ( t ) = ( \frac{μ}{{y}_{\frac{x}{s}}} + m ) * {V}_{{t}_{F}} {X}_{{t}_{F}} * {e}^{μ ( t - {t}_{F} )}$$

Here, ms is the mass flow of the substrate, p is the specific growth rate of the strain, y_{x/s} is the yield of biomass per unit of substrate, and m designates glucose for cell maintenance. Lastly, V represents the cultivation volume and X represents the biomass concentration at a given time.

After 8 hours of exponential feeding, feed rate was set constant with media glucose concentration measured consistently using a glucose meter (CVSHealth, USA) and high-performance liquid chromatography (HPLC). The feeding rate was adjusted so that the concentration of glucose was less than 1 g/L.

To prevent isoprenyl acetate evaporation, a 20% (v/v) oleyl alcohol overlay was added at the time of induction. As in the small-scale experiments, the organic phase and aqueous phase were first separated by centrifuge (for 10 min and at 5,000 g), then the organic phase was analyzed via GC-FID to determine isoprenol and isoprenyl acetate concentration while the aqueous phase was used for measurements of optical density, acetate, ethanol as well as OD_{600 nm}.

### Isoprenyl Acetate Toxicity Assay

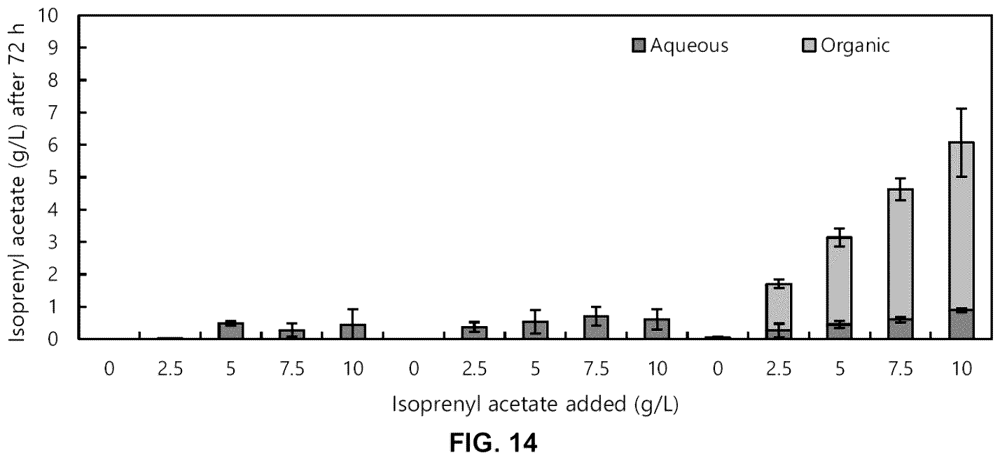

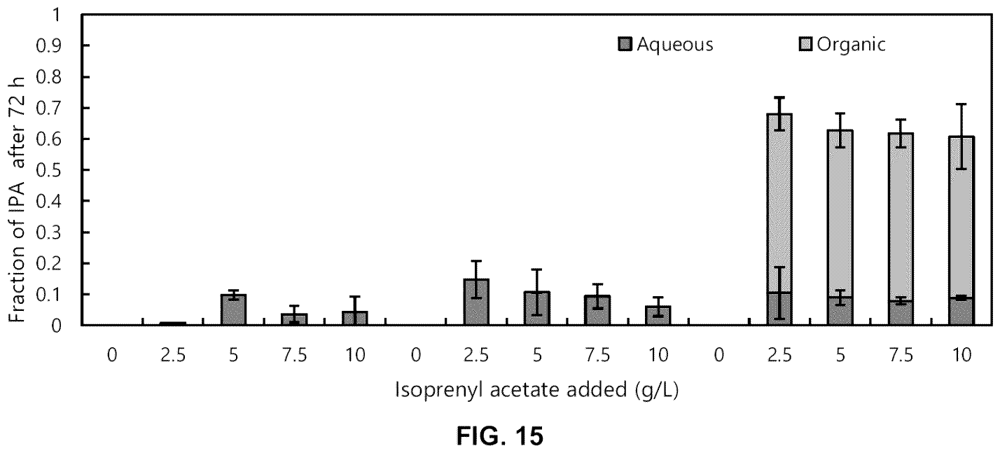

The relative differences in OD₆₀₀ with and without overlay provided strong evidence of isoprenyl acetate toxicity to E. coli DH1 as in the case of isoprenol. The relative difference in ester vapor pressure and water solubility compared to their respective alcohols as well as the presence of a strong banana/green apple odor that aligned with the organoleptic characterisation of isoprenyl acetate further suggested product losses due to evaporation. A simple assay was conducted to assess the toxicity of isoprenyl acetate on E. coli DH1 by titrating concentrations ranging between 0 g/L and 10 g/L, which is near the solubility limit of isoamyl acetate in water, into inoculated cultures in M9-MOPS as depicted as a fraction of remaining (FIG. 14) and total remaining (FIG. 15) isoprenyl acetate.

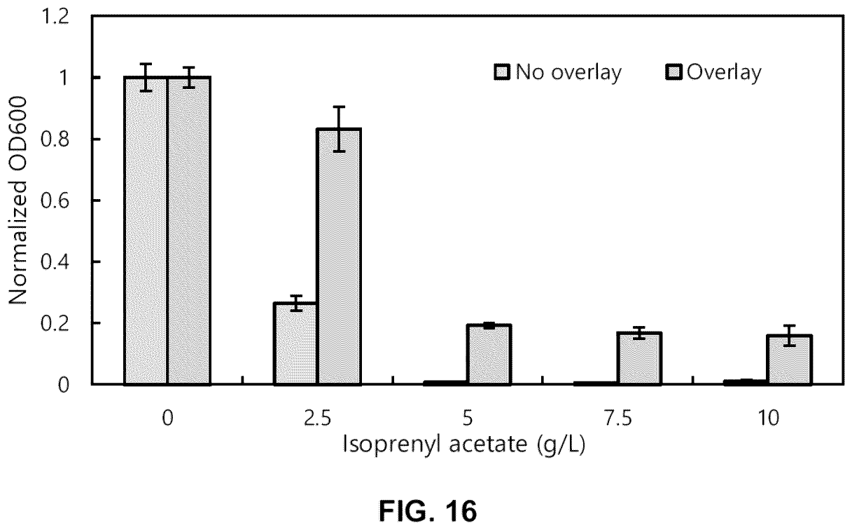

As depicted in FIG. 16, isoprenyl acetate is clearly deleterious to E. coli growth, resulting in a 57% decrease in final OD₆₀₀ between cultures with and without overlay in the 2.5 g/L condition over 72 hours. Growth in cultures above 2.5 g/L isoprenyl acetate was not observed without overlay, though those with overlay suffered significant growth inhibition, ultimately reaching an OD₆₀₀ of 20% of cultures without ester addition. In terms of final ester concentration, cultures without overlay demonstrated near complete ester loss over 72 hours due to evaporation. Those with overlay maintained approximately 60-70% of initial isoprenyl acetate. Importantly, these data also indicated a partitioning coefficient of 5 between oleyl alcohol and water while maintaining clear fractionation regardless of concentration. Overall, the determination of an LD₅₀ proved problematic in cultures without an overlay due to the unique balance between toxicity and significant evaporative losses. Nonetheless, these data conclusively support that isoprenyl acetate is not only toxic to E. coli but lends to significant evaporative losses without overlay or condensation.

### Quantification of Alcohols and Esters by Chromatography

Upon sampling, organic and aqueous phases were separated by centrifugation at 13,000 g for 5 minutes. The use of an organic solvent like oleyl alcohol demanded 1:100 dilution in ethyl acetate prior to GC loading. For the isoprenyl acetate quantification from organic phase, 10 μL of oleyl alcohol was added to 990 μL ethyl acetate containing 1-butanol (30 mg/L) as an internal standard. Conversely, a fraction of isolated culture aqueous phase (250 μL) was mixed in a 1:1 ratio with ethyl acetate and vortexed for 10 minutes at 3000 rpm (Scientific Industries INC, USA). Mixed phases were then centrifuged 13,000 g for 5 minutes with the organic phase diluted 1:5 in fresh ethyl acetate containing 1-butanol (30 mg/L) to a final volume of 1 mL. For high titer biofermenter production of isoprenyl acetate, organic, and aqueous phases were further diluted.

For the quantification of isoprenyl acetate, 1 μL of diluted samples was analyzed by GC-mass spectrometry (GCMS, Agilent, USA) and FID (GC-FID, Thermo Focus, USA) equipped with a DB-WAX column (15 m, 0.32 mm inner diameter, 0.25 m film thickness, Agilent, USA). The temperature program on the oven was set as follows: initiation at 50° C. and held for 1 min, a ramp of 15° C./min to 100° C. and held 1 min, a ramp of 30° C./min to 230° C. and held at 230° C. for 1 min.

### Quantification of Metabolites

Interest in acetate pathway rebalancing demanded quantification of acetate accumulation in our culture media. As a result, a further 500 μL of aqueous phase was separated for analysis by HPLC (Agilent 1200 Series, Santa Clara, CA) fitted with an Aminex HPX-87H column (Bio-Rad, Richmond, CA). Samples were initially filtered through a 0.45 μM syringe and scrutinized for residual organic phase, then 10.0 μL was injected with a 0.005 M H₂SO₄ mobile phase. The flow rate was 0.6 mL/min with the column held at 65° C. and samples were analyzed using a relative index detector at 45° C.

### Organic Synthesis of Analytical Ester Standards

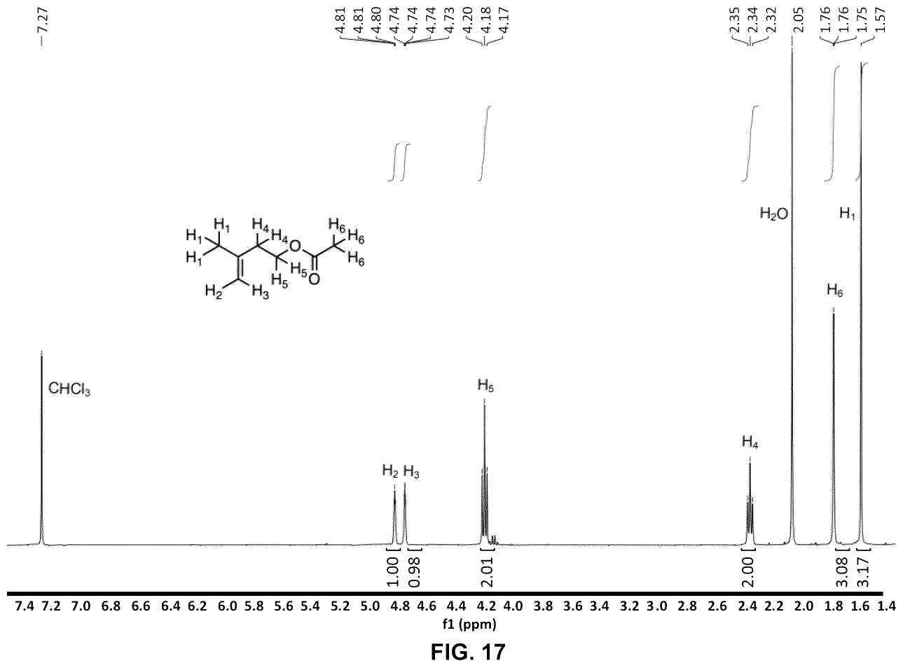

A synthesis of isoprenyl acetate was conducted as described (8). In brief, isoprenol (2.0 mL, 20 mM) was mixed with catalytic 4-dimethylaminopyridine (100 mg, 0.82 mM), and acetic anhydride (4.0 mL, 42.3 mM) at room temperature overnight. The reaction was quenched by addition of DI H₂O. The organic phase was extracted by addition of diethyl ether, then washed three times with saturated aqueous Na₂HCO₃, separated, and dried over anhydrous Na₂MgSO₄. The product was isolated by silica gel chromatography using a mixture of hexane and diethyl ether (9:1). Concentration under reduced pressure yielded a highly pure product as verified by GC-MS and 1H NMR (FIG. 17).

### Isoprenyl Acetate Production by Continuous Off-Gas Capture

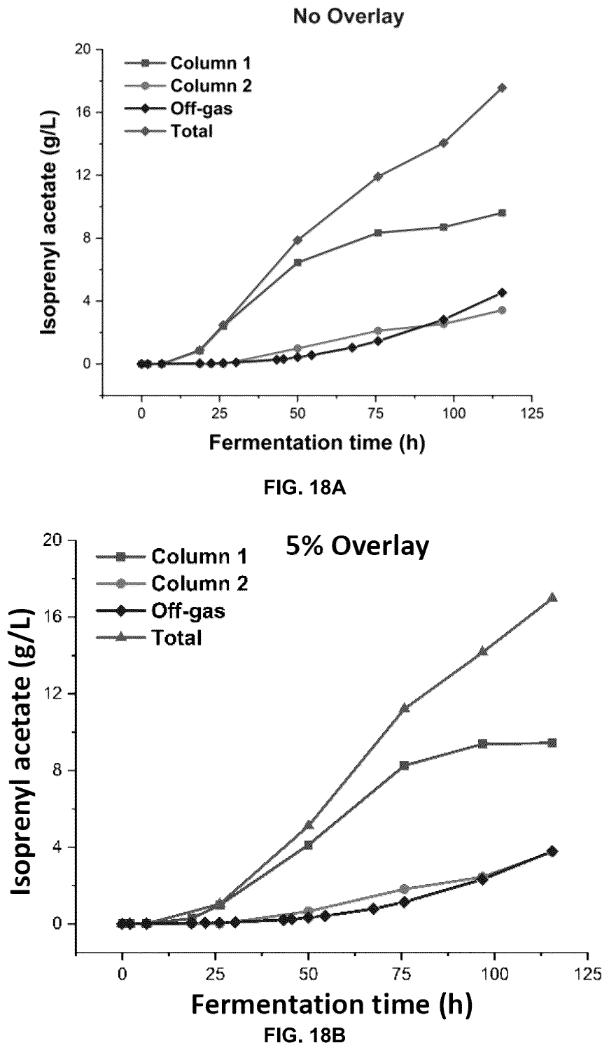

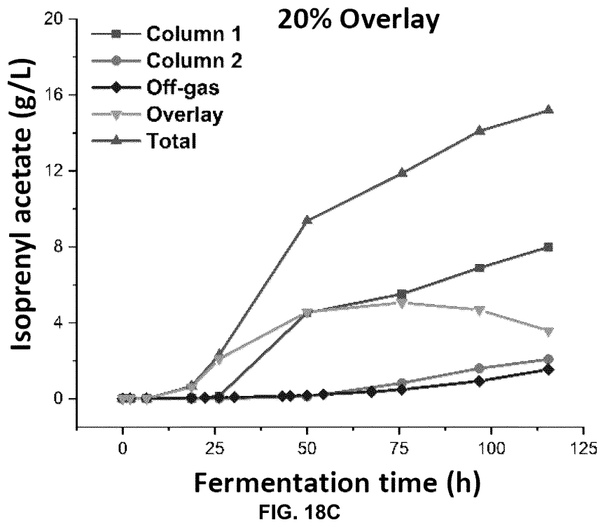

A comparative experiment is performed to evaluate the efficiency of product removal/recovery in 2 L scale using different overlay concentrations. Under all conditions, the product is continuously stripped from the fermenter to the off-gas and captured in two columns in series filled with chilled Durasyn® 164 (INEOS, Houston, TX). No overlay is added in the first reactor, and 5% and 20% (v/v) oleyl alcohol are added to the second and third fermentors. Final isoprenyl acetate concentrations at 0%, 5% and 20% overlay are 17.6, 17.0 and 15.2 g/L, with off-gas recovery of 74.2%, 77.7% and 66.3% respectively (FIGS. 18A to 18C).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

We claim:

1. A method for producing an olefinic ester, the method comprising: (a) providing a host cell capable of producing olefinic ester; and (b) culturing the host cell to produce olefinic ester.

2. The method of claim 1, wherein the olefinic ester is isoprenyl acetate.

3. The method of claim 2, comprising: (c) recovering the isoprenyl acetate.

4. The method of claim 3, comprising: (d) (i) deacetylating the isoprenyl acetate into isoprene, and/or (ii) hydrolyzing isoprenyl acetate into isoprenol and dehydrating the isoprenol into isoprene.

5. The method of claim 4, comprising: (e) converting the isoprene into 1,4-dimethylcyclooctane (DMCO).

6. A genetically modified host cells comprising a heterologous mevalonate pathway with the isopentenyl diphosphate (IPP) bypass and a promiscuous phosphatase to generate isoprenol.

7. The genetically modified host cells of claim 6, further comprising an alcohol acyltransferase (ATF1), or homologous enzyme thereof, capable of catalyzing an acetylation of isoprenol and acetyl-CoA to generate isoprenyl acetate.

8. The genetically modified host cells of claim 7, wherein the ATF1, or homologous enzyme thereof, comprises an amino acid sequence having at least about 70% amino acid sequence identity with SEQ ID NO:3, SEQ ID NO:4, SEQ ID NO:5, or SEQ ID NO:6.

9. The genetically modified host cells of claim 7, wherein the ATF1, or homologous enzyme thereof, comprises one or more of the conserved amino acid residues indicated by an asterisk and/or period shown in FIG. 5.


SEQUENCE LISTING (9 sequences)

SEQ ID NO: 1 - protein, 396 aa, Saccharomyces cerevisiae
MTVYTASVTAPVNIATLKYWGKRDTKLNLPTNSSISVTLSQDDLRTLTSAATAPEFERDTLWLNGEPHSIDNERTQNCLRDLRQLRKEMESKDASLPTLSQWKLHIVSENNFPTAAGLASSAAGFAALVSAIAKLYQLPQSTSEISRIARKGSGSACRSLFGGYVAWEMGKAEDGHDSMAVQIADSSDWPQMKACVLVVSDIKKDVSSTQGMQLTVATSELFKERIEHVVPKRFEVMRKAIVEKDFATFAKETMMDSNSFHATCLDSFPPIFYMNDTSKRIISWCHTINQFYGETIVAYTFDAGPNAVLYYLAENESKLFAFIYKLFGSVPGWDKKFTTEQLEAFNHQFESSNFTARELDLELQKDVARVILTQVGSGPQETNESLIDAKTGLPKE

SEQ ID NO: 2 - protein, 327 aa, Saccharomyces cerevisiae
MVKSGKARAHTNIALIKYWGKADETYIIPMNNSLSVTLDRFYTETKVTFNPDFTEDCLILNGNEVNAKEKEKIQNYMNIVRDLAGNRLHARIESENYVPTAAGLASSASAYAALAAACNEALSLNLSDTDLSRLARRGSGSASRSIFGGFAEWEKGHDDLTSYAHGINSNGWEKDLSMIFVVINNQSKKVSSRSGMSLTRDTSRFYQYWLDHVDEDLNEAKEAVKNQDFQRLGEVIEANGLRMHATNLGAQPPFTYLVQESYDAMAIVEQCRKANLPCYFTMDAGPQVKVLVEKKNKQAVMEQFLKVFDESKIIASDIISSGVEIIK

SEQ ID NO: 3 - protein, 521 aa, Saccharomyces cerevisiae
MNEIDGKSQGPVQQECLKEMIQNRHARCMGSVEDLYVALNREHLYRNFCTYGELSDYCSRDQLTLALREICLKNPTLLHIVLPTRWPNHENYYRSSEYYSRPHPVHDYISVLQELKLSGVVLNEQPEYSAVMKQILEEFKNSEGSYNAGVFKLSTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLKGCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEENFEWLTVI

SEQ ID NO: 4 - protein, 535 aa, Saccharomyces cerevisiae
MEDIEGYEPHITQELIDRGHARRMGHLENYFAVLSRQKMYSNFTVYAELNKGVNKRQLMLVLKVLLQKYSTLAHTIIPKHYPHHEAYYSSEEYLSKPFPQHDFIKVISHLEFDDLIMNNQPEYREVMEKISEQFKKDDFKVTNRLIELISPVIIPLGNPKRPNWRLICLPGKDTDGFETWKNFVYVTNHCGSDGVSGSNFFKDLALLFCKIEEKGFDYDEEFIEDQVIIDYDRDYTEISKLPKPITDRIDYKPALTSLPKFFLTTFIYEHCNFKTSSESTLTARYSPSSNANASYNYLLHFSTKQVEQIRAQIKKNVHDGCTLTPFIQACFLVALYRLDKLFTKSLLEYGFDVAIPSNARRFLPNDEELRDSYKYGSNVGGSHYAYLISSFDIPEGDNDKFWSLVEYYYDRFLESYDNGDHLIGLGVLQLDFIVENKNIDSLLANSYLHQQRGGAIISNTGLVSQDTTKPYYVRDLIFSQSAGALRFAFGLNVCSTNVNGMNMDMSVVQGTLRDRGEWESFCKLFYQTIGEFASL

SEQ ID NO: 5 - protein, 525 aa, Saccharomyces cerevisiae
MNEIDEKNQAPVQQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 6 - protein, 545 aa, Saccharomyces carlsbergensis
METEESQFSSITKIINPKTLMNTYSEKTSLVQDECLVKMIQNGHSRRMGSVEDLYAALNRQKLYRNFSTYSELNDYCTKDQLALALRNICLKNPTLLHIVLPARWPDHEKYYLSSEYYSQPRPKHDYISVLPELKLDGVILNEQPEHNALMKQILEEFANSNGSYTAKIFKLTTALTIPYTGPTSPTWRLICLPEEDDTNKWKKFIFVSNHCMCDGRSSIHFFQDLRDELNNIKTLPKKLDYIFEYEKDYQLLRKLPEPIENMIDFRPPYLFIPKSLLSGFIYSHLRFSSKGVCTRMDEIEKSDEIVTEIINISPSEFQKIRTKIKLNIPGKCTITPFLEVCWFVTLHKWGKFFKPLKFEWLTDVFIPADCRSLLPEDEEVRAMYRYGANVGFVDFTPWISKFNMNDSKENFWPLIAHYHEVISGAIKDKKHLNGLGFNIQSLVQKYVNIDKVMRDRALGKSRGGTLLSNVGMFHQSEETEHKYRIRDLAFGQFQGSWHQAFSLGVSSTNVKGMNILISSTKNVVGSQELLEELCAMYKALLLNP

SEQ ID NO: 7 - protein, 650 aa, Escherichia coli
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAM

SEQ ID NO: 8 - protein, 650 aa, Salmonella enterica
MSQTHKHAIPANIADRCLINPEQYETKYKQSINDPDTFWGEQGKILDWITPYQKVKNTSFAPGNVSIKWYEDGTLNLAANCLDRHLQENGDRTAIIWEGDDTSQSKHISYRELHRDVCRFANTLLDLGIKKGDVVAIYMPMVPEAAVAMLACARIGAVHSVIFGGFSPEAVAGRIIDSSSRLVITADEGVRAGRSIPLKKNVDDALKNPNVTSVEHVIVLKRTGSDIDWQEGRDLWWRDLIEKASPEHQPEAMNAEDPLFILYTSGSTGKPKGVLHTTGGYLVYAATTFKYVFDYHPGDIYWCTADVGWVTGHSYLLYGPLACGATTLMFEGVPNWPTPARMCQVVDKHQVNILYTAPTAIRALMAEGDKAIEGTDRSSLRILGSVGEPINPEAWEWYWKKIGKEKCPVVDTWWQTETGGFMITPLPGAIELKAGSATRPFFGVQPALVDNEGHPQEGATEGNLVITDSWPGQARTLFGDHERFEQTYFSTFKNMYFSGDGARRDEDGYYWITGRVDDVLNVSGHRLGTAEIESALVAHPKIAEAAVVGIPHAIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAM

SEQ ID NO: 9 - protein, 652 aa, Salmonella enterica
MSQTHKHAIPANIADRCLINPEQYETKYKQSINDPDTFWGEQGKILDWITPYQKVKNTSFAPGNVSIKWYEDGTLNLAANCLDRHLQENGDRTAIIWEGDDTSQSKHISYRELHRDVCRFANTLLDLGIKKGDVVAIYMPMVPEAAVAMLACARIGAVHSVIFGGFSPEAVAGRIIDSSSRLVITADEGVRAGRSIPLKKNVDDALKNPNVTSVEHVIVLKRTGSDIDWQEGRDLWWRDLIEKASPEHQPEAMNAEDPLFILYTSGSTGKPKGVLHTTGGYLVYAATTFKYVFDYHPGDIYWCTADVGWVTGHSYLLYGPLACGATTLMFEGVPNWPTPARMCQVVDKHQVNILYTAPTAIRALMAEGDKAIEGTDRSSLRILGSVGEPINPEAWEWYWKKIGKEKCPVVDTWWQTETGGFMITPLPGAIELKAGSATRPFFGVQPALVDNEGHPQEGATEGNLVITDSWPGQARTLFGDHERFEQTYFSTFKNMYFSGDGARRDEDGYYWITGRVDDVLNVSGHRLGTAEIESALVAHPKIAEAAVVGIPHAIKGQAIYAYVTLNHGEEPSPELYAEVRNWVRKEIGPLATPDVLHWTDSLPKTRSGKIMRRILRKIAAGDTSNLGDTSTLADPGVVEKPLEEKQAIAMPS